(19) United States
(12) Patent Application Publication
(10) Pub. No.: US 2006/0002962 A1
(43) Pub. Date: Jan. 5, 2006

(54) Method of treating allergy and infection by eliciting an IGA antibody response

(75) Inventors: Henrik Hugo Jacobi, Vedbaek (DK); Jens Kildsgaard, Holte (DK)

Correspondence Address: BIRCH STEWART KOLASCH & BIRCH, PO BOX 747, FALLS CHURCH, VA, 22040-0747, US

(73) Assignee: Alk-Abello A/S, Horsholm (DK)

(21) Appl. No.: 11/139,623

(22) Filed: May 31, 2005

Related U.S. Application Data
(60) Provisional application No. 60/574,941, filed on May 28, 2004.

(30) Foreign Application Priority Data
May 28, 2004 (DK) PA2004 00853
Jan. 7, 2005 (DK) PA2005 00033

(51) Int. Cl. A61K 39/385

(52) U.S. Cl. 424/275.1

(57) ABSTRACT

The invention relates to a method of preventing or treating an allergy to or an infection by a mucosal antigenic agent in a subject comprising a) subjecting the subject to a priming treatment by means of parenteral or adjuvant-facilitated mucosal administration to activate the systemic immune system, and b) subjecting the subject to Specific Allergy Vaccination (SAV) by means of non-gastrointestinal mucosal administration to elicit an antigenic agent specific Ig antibody response in the mucosa of the subject.

### TECHNICAL FIELD

The present invention relates to a method of preventing or treating an allergy to or an infection by a mucosal antigenic agent in a subject.

### BACKGROUND OF THE INVENTION

Allergy is a major health problem in countries where Western lifestyle is adapted. Furthermore, the prevalence of allergic disease is increasing in these countries. Although allergy in general may not be considered a life-threatening disease, asthma annually causes a significant number of deaths. An exceptional prevalence of about 30% in teenagers conveys a substantial loss in quality of life, working days and money, and warrants a classification among major health problems in the Western world.

Allergy is a complex disease. Many factors contribute to the sensitisation event. Among these is the susceptibility of the individual defined by an as yet insufficiently understood interplay between several genes. Another important factor is allergen exposure above certain thresholds. Several environmental factors may be important in the sensitisation process including pollution, childhood infections, parasite infections, intestinal microorganisms, etc. Once an individual is sensitised and the allergic immune response established, the presence of only minute amounts of allergen is efficiently translated into symptoms.

The natural course of allergic disease is usually accompanied by aggravation at two levels. Firstly, a progression of symptoms and disease severity, as well as disease progression, for example from hay fever to asthma.

Secondly, dissemination in offending allergens most often occurs resulting in allergic multi-reactivity. Chronic inflammation leads to a general weakening of the mucosal defense mechanisms resulting in unspecific irritation and eventually destruction of the mucosal tissue. Infants may become sensitised primarily to foods, i.e. milk, resulting in eczema or gastrointestinal disorders; however, most often they outgrow these symptoms spontaneously. These infants are at risk of developing inhalation allergy later in their lives.

The most important allergen sources are found among the most prevalent particles of a certain size in the air we breathe. These sources are remarkably universal and include grass pollens and house dust mite faecal particles, which together are responsible for approximately 50% of all allergies. Of global importance are also animal dander, i.e. cat and dog dander, other pollens, such as mugwort pollens, and micro-fungi, such as Alternaria. On a regional basis yet other pollens may dominate, such as birch pollen in Northern and Central Europe, ragweed in the Eastern and Central United States, and Japanese cedar pollen in Japan. Insects, i.e. bee and wasp venoms, and foods each account for approximately 2% of all allergies.

Allergy, i.e. type I hyper-sensitivity, is caused by an inappropriate immunological reaction to foreign non-pathogenic substances. Important clinical manifestations of allergy include asthma, hay fever, eczema, and gastro intestinal disorders. The allergic reaction is prompt and peaks within 20 minutes upon contact with the offending allergen. Furthermore, the allergic reaction is specific in the sense that a particular individual is sensitised to particular allergen(s), whereas the individual does not necessarily show an allergic reaction to other substances known to cause allergic disease. The allergic phenotype is characterized by a pronounced inflammation of the mucosa of the target organ and by the presence of allergen specific antibody of the IgE class in the circulation and on the surfaced of mast-cells and basophils.

An allergic attack is initiated by the reaction of the foreign allergen with allergen specific IgE antibodies, when the antibodies are bound to high affinity IgE specific receptors on the surface of mast-cells and basophils. The mast-cells and basophils contain preformed mediators, i.e. histamine, tryptase, and other substances, which are released upon cross-linking of two or more receptor-bound IgE antibodies. IgE antibodies are cross-linked by the simultaneous binding of one allergen molecule. It therefore follows that a foreign substance having only one antibody binding epitope does not initiate an allergic reaction. The cross-linking of receptor bound IgE on the surface of mast-cells also leads to release of signaling molecules responsible for the attraction of eosinophils, allergen specific T-cells, and other types of cells to the site of the allergic response. These cells in interplay with allergen, IgE and effector cells, lead to a renewed flash of symptoms occurring 12-24 hours after allergen encounter (late phase reaction).

Allergy disease management comprises diagnosis and treatment including prophylactic treatments. Diagnosis of allergy is concerned with by the demonstration of allergen specific IgE and identification of the allergen source. In many cases a careful anamnesis may be sufficient for the diagnosis of allergy and for the identification of the offending allergen source material. Most often, however, the diagnosis is supported by objective measures, such as skin prick test, blood test, or provocation test.

The therapeutic options fall in three major categories. The first opportunity is allergen avoidance or reduction of the exposure. Whereas allergen avoidance is obvious e.g. in the case of food allergens, it may be difficult or expensive, as for house dust mite allergens, or it may be impossible, as for pollen allergens. The second and most widely used therapeutic option is the prescription of classical symptomatic drugs like anti-histamines and steroids. Symptomatic drugs are safe and efficient; however, they do not alter the natural cause of the disease, neither do they control the disease dissemination. The third therapeutic alternative is specific allergy vaccination that in most cases reduces or alleviates the allergic symptoms caused by the allergen in question.

Conventional specific allergy vaccination is a causal treatment for allergic disease. It interferes with basic immunological mechanisms resulting in persistent improvement of the patients' immune status. Thus, the protective effect of specific allergy vaccination extends beyond the treatment period in contrast to symptomatic drug treatment. Some patients receiving the treatment are cured, and in addition, most patients experience a relief in disease severity and symptoms experienced, or at least an arrest in disease aggravation. Thus, specific allergy vaccination has preventive effects reducing the risk of hay fever developing into asthma, and reducing the risk of developing new sensitivities.

The immunological mechanism underlying successful allergy vaccination is not known in detail. A specific immune response, such as the production of antibodies against a particular pathogen, is known as an adaptive immune response. This response can be distinguished from the innate immune response, which is an unspecific reaction towards pathogens. An allergy vaccine is bound to address the adaptive immune response, which includes cells and molecules with antigen specificity, such as T-cells and the antibody producing B-cells. B-cells cannot mature into antibody producing cells without help from T-cells of the corresponding specificity. T-cells that participate in the stimulation of allergic immune responses are primarily of the Th2 type. Establishment of a new balance between Th1 and Th2 cells has been proposed to be beneficial and central to the immunological mechanism of specific allergy vaccination. Whether this is brought about by a reduction in Th2 cells, a shift from Th2 to Th1 cells, or an up-regulation of Th1 cells is controversial. Recently, regulatory T-cells have been proposed to be important for the mechanism of allergy vaccination. According to this model regulatory T-cells, i.e. Th3 or Tr1 cells, down-regulate both Th1 and Th2 cells of the corresponding antigen specificity. In spite of these ambiguities it is generally believed that an active vaccine must have the capacity to stimulate allergen specific T-cells, preferably TH1 cells.

Specific allergy vaccination is, in spite of its virtues, not in widespread use, primarily for two reasons. One reason is the inconveniences associated with the traditional vaccination programme that comprises repeated vaccinations i.a. injections over a several months. The other reason is, more importantly, the risk of allergic side reactions. Ordinary vaccinations against infectious agents are efficiently performed using a single or a few high dose immunizations. This strategy, however, cannot be used for allergy vaccination since a pathological immune response is already ongoing.

Conventional specific allergy vaccination is therefore carried out using multiple subcutaneous immunizations applied over an extended time period. The course is divided in two phases, the up dosing and the maintenance phase. In the up dosing phase increasing doses are applied, typically over a 16-week period, starting with minute doses. When the recommended maintenance dose is reached, this dose is applied for the maintenance phase, typically with injections every six weeks. Following each injection the patient must remain under medical attendance for 30 minutes due to the risk of anaphylactic side reactions, which in principle although extremely rare could be life-threatening. In addition, the clinic should be equipped to support emergency treatment. There is no doubt that a vaccine based on a different route of administration would eliminate or reduce the risk for allergic side reactions inherent in the current subcutaneous based vaccine as well as would facilitate a more widespread use, possibly even enabling self vaccination at home.

Attempts to improve vaccines for specific allergy vaccination have been performed for over 30 years and include multifarious approaches. Several approaches have addressed the allergen itself through modification of the IgE reactivity.

WO 9730728 discloses a pharmaceutical composition for treating e.g. allergy comprising an immune-stimulating complex and a mucos-targetting molecule. The composition provides a high IgA antibody titre when administered to the mucosa.

WO 200151082 discloses a pharmaceutical composition for treating e.g. allergy comprising one or more IgA inducing allergens, e.g. from a plant, a glycolipid adjuvant and a sublingually administrable diluent, excipient or carrier. The composition produces a mucosal immune response.

Ogra et al. (2001) mentions that mucosal administration of vaccines induce immune responses in most external surfaces, and that it is possible to manipulate the mucosal immune system to induce systemic tolerance against environmental microbial antigens, dietary antigens and autoantigens to treat infectious diseases and autoimmune diseases. Rectal, oral, nasal, genital, systemic and transcutaneous administration is mentioned.

Sabbah (1998) discloses allergen specific immunotherapy using sublingual administration (SLIT), wherein the allergen is a pollen allergen, such as grass and ragweed.

Wiedermann et al. (1998) relates to a model of aerosol inhalation leading to allergic sensitisation in BALB/c mice. One group of mice were aerosolised with Bet v 1 allergen and adjuvant cholera toxin B. IgA antibodies were detected in bronchial lavage of the treated mice.

Holt (1998) discloses the development of oral tolerance in humans, which typically occurs in early postnatal life and is likely to play an important role in limiting the development of food allergy. Inert protein antigens fed to immunological individuals elicit initial immune responses which are recognised as Th2-polarised, but with continuing exposure these responses are self-limiting, and are supplanted by a state of antigen-specific tolerance.

Taudorf et al. (1994) discusses two potential mechanisms for oral immunotherapy, viz. induction of mucosal secretory IgA response and induction of systemic hyporesponsiveness (oral tolerance). Birch pollen antigens in enterocoated capsules were given to a group of persons. The IgA level in saliva and tears were measured, and no explanation for the beneficial effect of oral immunotherapy was found.

Horak et al. (Int. Archs Allergy appl. Immun. 84: 74-78 (1987)) discloses a combination treatment of grass pollen allergy comprising a) treatment with tyrosine-adsorbed glutaraldehyde-modified grass pollen extract (mixture of 12 grasses) allergoid followed by b) oral extension treatment using encapsulated, particulate enteric-coated grass pollen extract allergoid.

Trede et al. (Allergy, 1989, 44, 272-280) discloses a combination treatment of grass pollen allergy comprising a) a parenteral pre-seasonal priming treatment with tyrosine-allergoid grass pollen and b) capsules containing an enteric-coated mixture of 13 common grass allergens.

Wheeler et al. (Int. Archs Allergy appl. Immun. 83: 354-358 (1987)) discloses a combination treatment of grass pollen allergy comprising a) subcutaneous injection with grass pollen extract in physiological phosphate-buffered saline (PBS) followed by b) oral administration of enteric-coated allergen grass extract containing a mixture of 12 grasses encapsulated in Eudragit (a methacrylic acid, methyl methacrylate copolymer).

Henderson et al. (Int. Archs Allergy appl. Immun. 79: 66-71 (1986)) discloses mouse experiments, wherein mice are subjected to a combination treatment comprising priming by intragastrical administration followed by intraperitoneal administration either with or without alum. Another combination treatment protocol comprises priming by intraperitoneal or subcutaneous administration followed by intragastrical administration. Intragastrical administration consisted in 0.5 ml of a grass pollen allergen extract in physiological phosphate-buffered saline (PBS) given via an oral dosing needle. The formulation used for intraperitoneal and subcutaneous administration was prepared by dissolving the extract in PBS, and when using alum as adjuvant mixing the extract solution with a solution of alum.

Cox et al. (Int. Archs Allergy appl. Immun. 75: 126-131 (1984)) discloses a treatment method involving parenteral administration by injection with antigen in solution and in particulate form (antigen conjugated to polyacrylamide) followed by gastric intubation of antigen in solution or antigen in particulate form.

The methods according to Horak et al., Trede et al., Wheeler et al., Henderson et al. and Cox et al. all differ from the present method of treatment in that they involve administration via an intestinal mucosal route.

WO 02/45741 discloses a method of raising an immune response in a mammal comprising mucosal administration of a first immunogenic composition followed by the parenteral administration of a second immunogenic composition. The first immunogenic composition may be adjuvanted. The antigenic agent is Helicobacter pylori CagA and NAP.

The object of the present invention is to provide a method of preventing or treating an allergy or an infection, which is effective and easy to carry out.

### SUMMARY OF THE INVENTION

This object is obtained by the method of the invention, which relates to a method of preventing or treating an allergy to or an infection by a mucosal antigenic agent in a subject comprising
a) subjecting the subject to a priming treatment by means of parenteral or adjuvant-facilitated mucosal administration of the antigenic agent to activate the systemic immune system, and
b) subjecting the subject to Specific Allergy Vaccination (SAV) by means of non-gastrointestinal mucosal administration of the antigenic agent to elicit an antigenic agent specific Ig antibody response in the mucosa of the subject.

The invention is based on the surprising finding that it is possible to elicit a strong mucosal IgA and IgM immune response by means of mucosal, e.g. oromucosal, vaccination, and that such a vaccination is an effective treatment of allergies and infections, wherein the action of the antigenic agent is effected via contact with the mucosa. It is believed that the mechanism of action of this treatment is that the IgA and IgM bind to the antigenic agent thereby neutralising it.

The invention is further based on the finding that a prerequisite for obtaining a generation of the above mentioned IgA and IgM immune response is that the patient prior to the mucosal vaccination has been subjected to a priming treatment, wherein an activation of the systemic immune system has been effected.

Also, the present invention is based on the surprising discovery that carrying out the SAV treatment step of the method of the invention by means of non-gastrointestinal mucosal administration is more effective than gastrointestinal mucosal administration.

As mentioned above, conventional vaccination is not convenient due to the need for a vaccination programme involving many injections over an extended period of time. A further advantage of the method of the present invention is that compared to conventional parenteral vaccination, the number of required parenteral administrations is reduced significantly. For example, the method of the invention has provided a possibility to conduct only the up-dosing phase of the vaccination programme, or even only the first few administrations, by means of parenteral administration, and then to conduct the maintenance phase, or the rest of the up-dosing phase and the maintenance phase, respectively, by means of mucosal administration.

The present invention further relates to the following aspects:

A vaccine for preventing or treating an allergy to or an infection by a mucosal antigenic agent in a subject having one or more antibodies specific for the antigenic agent by means of non-gastrointestinal mucosal administration to elicit an Ig antibody response in the mucosa of the subject.

Use of an allergen for preparing vaccine for preventing or treating an allergy to or an infection by a mucosal antigenic agent in a subject having one or more antibodies specific for the antigenic agent by means of non-gastrointestinal mucosal administration to elicit an Ig antibody response in the mucosa of the subject.

A method of preventing or treating an allergy to or an infection by a mucosal antigenic agent in a subject comprising
a) subjecting the subject to a priming treatment by means of oromucosal administration to activate the mucosal immune system, and
b) subjecting the subject to Specific Allergy Vaccination (SAV) by means of parenteral or adjuvant-facilitated mucosal administration to elicit an antigenic agent specific Ig antibody response in the mucosa of the subject.

### SHORT DESCRIPTION OF THE FIGURES

FIG. 1 shows the level of serum IgG₁ in mice subjected to 3 i.p. injections followed by six weeks of Sublingual Immunotherapy at 4 doses.

FIG. 2 shows the level of serum IgG₂ₐ in mice subjected to 3 i.p. injections followed by six weeks of Sublingual Immunotherapy at 4 doses.

FIG. 3 shows the level of serum IgG in mice subjected to 3 i.p. injections followed by six weeks of Sublingual Immunotherapy at 4 doses.

FIG. 4 shows the level of serum IgE in mice subjected to 3 i.p. injections followed by six weeks of Sublingual Immunotherapy at 4 doses.

FIG. 5 shows the level of BAL IgA in mice subjected to 3 i.p. injections followed by six weeks of Sublingual Immunotherapy at 2, 4 and 6 weeks and at 4 doses.

FIG. 6 shows the level of NAL IgA in mice subjected to 3 i.p. injections followed by six weeks of Sublingual Immunotherapy at 2, 4 and 6 weeks and at 4 doses.

FIG. 7 shows the inhibition of specific serum IgE by specific IgA for BAL containing a) a high level of IgA, b) a low level of IgA, c) no IgA (positive control), and d) no IgA and no serum (negative control).

FIG. 8 shows the T cell proliferation for mice treated with Phl p extract (parenteral plus SLIT treatment) or buffer at three concentrations.

FIG. 9 shows the cytokine production (five different cytokines) for mice treated with Phl p extract (parenteral plus SLIT treatment) or buffer.

FIG. 10 shows the level of specific serum IgA in mice subjected to 3 i.p. injections followed by six weeks of Sublingual Immunotherapy (SLIT) or peroral treatment (PO).

FIG. 11 shows the level of specific BAL IgA in mice subjected to 3 i.p. injections followed by six weeks of Sublingual Immunotherapy (SLIT) or peroral treatment (PO).

FIG. 12A-C show serum levels of Phl p specific total IgG, IgG1 and IgG2a in mice that have been treated with SLIT for six weeks.

FIG. 12 D-F show serum levels of Phl p specific total IgG, IgG1 and IgG2a in mice subjected to an identical administration of SLIT followed by one i.p. immunisation with Phl p extract (5 kSQ/alum).

FIG. 13A shows serum levels of Phl p specific IgE in mice that have been treated with SLIT for six weeks.

FIG. 13B shows serum levels of Phl p specific IgE in mice that have been treated with SLIT followed by one i.p. immunisation with Phl p extract.

FIG. 14 shows Phl p-specific IgE levels in sera of SLIT treated (hatched lines) and buffer treated control mice (solid lines).

FIG. 15 shows Phl p-specific IgA levels in BAL of SLIT treated and buffer treated control mice.

FIG. 16 shows the proliferation of spleen cells from mice treated with Phl p SLIT.

FIGS. 17A and 17B show the proliferation and cytokine production, respectively, of spleen cells from mice treated with Phl p SLIT followed by one immunization.

FIGS. 18A and 18B show the proliferation of spleen cells from mice treated with Phl p SLIT for three and six weeks, respectively, followed by one immunization.

FIG. 19 shows the proliferation of spleen cells from mice treated with different doses of Phl p SLIT followed by one immunization.

### DETAILED DESCRIPTION OF THE INVENTION

Mucosal Antigenic Agent

Allergy:

For the allergy indication, the mucosal antigenic agent is an allergen, which comes into contact with the mucosa of the subject, including the mucosa of the respiratory system, the mucosa of the digestive system, the rectal mucosa and the genital mucosa.

In a preferred embodiment of the invention the allergen according to the present invention is any naturally occurring protein that has been reported to induce allergic, i.e. IgE mediated reactions upon their repeated exposure to an individual. Examples of naturally occurring allergens include pollen allergens (tree-, herb, weed-, and grass pollen allergens), insect allergens (inhalant, saliva and venom allergens, e.g. mite allergens, cockroach and midges allergens, hymenopthera venom allergens), animal hair and dandruff allergens (from e.g. dog, cat, horse, rat, mouse etc.), and food allergens. Important pollen allergens from trees, grasses and herbs are such originating from the taxonomic orders of Fagales, Oleales, Pinales and platanaceae including i.a. birch (Betula), alder (Alnus), hazel (Corylus), hornbeam (Carpinus) and olive (Olea), cedar (Cryptomeria and Juniperus), Plane tree (Platanus), the order of Poales including i.a. grasses of the genera Lolium, Phleum, Poa, Cynodon, Dactylis, Holcus, Phalaris, Secale, and Sorghum, the orders of Asterales and Urticales including i.a. herbs of the genera Ambrosia, Artemisia, and Parietaria. Other important inhalation allergens are those from house dust mites of the genus Dermatophagoides and Euroglyphus, storage mite e.g Lepidoglyphys, Glycyphagus and Tyrophagus, those from cockroaches, midges and fleas e.g. Blatella, Periplaneta, Chironomus and Ctenocepphalides, and those from mammals such as cat, dog and horse, venom allergens including such originating from stinging or biting insects such as those from the taxonomic order of Hymenoptera including bees (superfamily Apidae), wasps (superfamily Vespidea), and ants (superfamily Formicoidae). Important inhalation allergens from fungi are i.a. such originating from the genera Alternaria and Cladosporium.

In a more preferred embodiment of the invention the allergen is Bet v 1, Aln g 1, Cor a 1 and Car b 1, Que a 1, Cry j 1, Cry j 2, Cup a 1, Cup s 1, Jun a 1, Jun a 2, jun a 3, Ole e 1, Lig v 1, Pla l 1, Pla a 2, Amb a 1, Amb a 2, Amb t 5, Art v 1, Art v 2 Par j 1, Par j 2, Par j 3, Sal k 1, Ave e 1, Cyn d 1, Cyn d 7, Dac g 1, Fes p 1, Hol l 1, Lol p 1 and 5, Pha a 1, Pas n 1, Phl p 1, Phl p 5, Phl p 6, Poa p 1, Poa p 5, Sec c 1, Sec c 5, Sor h 1, Der f 1, Der f 2, Der p 1, Der p 2, Der p 7, Der m 1, Eur m 2, Gly d 1, Lep d 2, Blo t 1, Tyr p 2, Bla g 1, Bla g 2, Per a 1, Fel d 1, Can f 1, Can f 2, Bos d 2, Equ c 1, Equ c 2, Equ c 3, Mus m 1, Rat n 1, Apis m 1, Api m 2, Ves v 1, Ves v 2, Ves v 5, Dol m 1, Dil m 2, Dol m 5, Pol a 1, Pol a 2, Pol a 5, Sol i 1, Sol i 2, Sol i 3 and Sol i 4, Alt a 1, Cla h 1, Asp f 1, Bos d 4, Mal d 1, Gly m 1, Gly m 2, Gly m 3, Ara h 1, Ara h 2, Ara h 3, Ara h 4, Ara h 5 or shufflant hybrids from Molecular Breeding of any of these.

In the most preferred embodiment of the invention the allergen is grass pollen allergen or a dust mite allergen or a ragweed allergen or a cedar pollen or a cat allergen or birch allergen.

In yet another embodiment of the invention the fast dispersing solid dosage form comprises at least two different types of allergens either originating from the same allergic source or originating from different allergenic sources e.g. grass group 1 and grass group 5 allergens or mite group 1 and group 2 allergens from different mite and grass species respectively, weed antigens like short and giant ragweed allergens, different fungis allergens like alternaria and cladosporium, tree allergens like birch, hazel, hornbeam, oak and alder allergens, food allergens like peanut, soybean and milk allergens.

The allergen incorporated into the fast dispersing solid dosage form may be in the form of an extract, a purified allergen, a modified allergen, a recombinant allergen or a mutant of a recombinant allergen. An allergenic extract may naturally contain one or more isoforms of the same allergen, whereas a recombinant allergen typically only represents one isoform of an allergen. In a preferred embodiment the allergen is in the form of an extract. In another preferred embodiment the allergen is a recombinant allergen. In a further preferred embodiment the allergen is a naturally occurring low IgE-binding mutant or a recombinant low IgE-binding mutant.

Allergens may be present in equi-molar amounts or the ratio of the allergens present may vary preferably up to 1:20.

In a further embodiment of the invention the low IgE binding allergen is an allergen according to WO 99/47680 or WO02/40676 and in not yet published patent application “Allergen mutants” by ALK-Abelló A/S.

Infection:

For the infection indication, the antigenic agent is a microbial antigen, which comes into contact with the mucosa of the subject, including the mucosa of the respiratory system, the mucosa of the digestive system, the rectal mucosa and the genital mucosa.

In a preferred embodiment of the invention, the microbial agent is a virus, a bacteria, a fungus, a parasite or any part thereof.

Examples of microbial agents are Vibrio species, Salmonella species, Bordetella species, Haemophilus species, Toxoplasmosis gondii, Cytomegalovirus, Chlamydia species, Streptococcal species, Norwalk Virus, Escherischia coli, Helicobacter pylori, Helicobacter felis, Rotavirus, Neisseria gonorrhae, Neisseria meningiditis, Adenovirus, Epstein Barr Virus, Japanese Encephalitis Virus, Pneumocystis carini, Herpes simplex, Clostridia species, Respiratory Syncytial Virus, Klebsielia species, Shigella species, Pseudomonas aeruginosa, Parvovirus, Campylobacter species, Rickettsia species, Varicella zoster, Yersinia species, Ross River Virus, J. C. Virus, Rhodococcus equi, Moraxella catarrhalis, Borrelia burgdorferi, Pasteurella haemolytica, poliovirus, influenza virus, Vibrio cholerae and Salmonella enterica serovar Typhi.

Further examples of microbial agents are those, which prevent or reduce the symptoms of the following diseases: Influenza, Tuberculosis, Meningitis, Hepatitis, Whooping Cough, Polio, Tetanus, Diphtheria, Malaria, Cholera, Herpes, Typhoid, HIV, AIDS, Measles, Lyme disease, Travellers Diarrhea, Hepatitis A, B and C, Otitis Media, Dengue Fever, Rabies, Parainfluenza, Rubella, Yellow Fever, Dysentery, Legionnaires Disease, Toxoplasmosis, Q-Fever, Haemorrhegic Fever, Argentina Haemorrhagic Fever, Caries, Chagas Disease, Urinary Tract Infection caused by E. coli, Pneumoccoccal Disease, Mumps, and Chikungunya.

Allergy

Examples of allergies included in the scope of the present invention are all allergies caused by the allergens mentioned above.

Infection

Examples of infections included in the scope of the present invention are all infections caused by the microbial agents mentioned above.

Priming Treatment

The generation of the mucosal IgA and IgM immune response is dependent on the activation of the systemic immune system.

It is hypothesized that IgA is induced via TGF-β, which is believed to be the most important switch factor for B cells toward IgA production. It has been shown (Sohn et al. 2003) that the ligation of the complement C3 activation product iC3b to complement receptor receptor type 3 (the iC3b receptor) on antigen-presenting cells results in the sequential production of TGF-β2 and IL-10. The strongly increased IgA response seen in the experiments presented in the Examples is believed to depend on the presence of allergen specific antibodies, e.g. IgG, which forms aggregates consisting of allergen, antibody and complement fragments.

In one embodiment of the invention the priming treatment is carried out by parenteral administration. Parenteral administration includes intravenous, intramuscular, intraarticular, subcutaneous, intradermal, epicutaneous/transdermal and intraperitoneal administration. Vaccines for administration via injection may be formulated so as to be suitable for injection by needle or for needleless injection.

In another embodiment of the invention the priming treatment is carried out by adjuvant-facilitated mucosal administration. Mucosomal administration includes oral, nasal, vaginal, sublingual, ocular, rectal, urinal, intramammal, pulmonal, otolar (i.e. via the ear) or buccal administration, preferably buccal or sublingual administration. The mucosal vaccine may be in the form of a spray, an aerosol, a mixture, a suspension, a dispersion, an emulsion, a gel, a paste, a syrup, a cream, an ointment, implants (ear, eye, skin, nose, rectal, and vaginal), intramammary preparations, vagitories, suppositories, or uteritories.

Preparation of vaccines is generally well known in the art. The antigenic agent may suitably be mixed with excipients which are pharmaceutically acceptable and further compatible with the active ingredient. Examples of suitable excipients are water, saline, dextrose, glycerol, ethanol and the like as well as combinations thereof. The vaccine may additionally contain other substances such as wetting agents, emulsifying agents, buffering agents or adjuvants enhancing the effectiveness of the vaccine.

Vaccines may suitably be formulated with excipients normally employed for such formulations, e.g. pharmaceutical grades of mannitol, lactose, starch, magnesium stearate, sodium saccharine, cellulose, magnesium carbonate and the like.

The vaccines are administered in a way so as to be compatible with the dosage formulation and in such amount as will be therapeutically effective and immunogenic. The quantity of active component contained within the vaccine depends on the subject to be treated, i.a. the capability of the subject's immune system to respond to the treatment, the route of administration and the age and weight of the subject. In general the priming treatment may e.g. consist in a treatment protocol, which corresponds to the up-dosing protocol of conventional parenteral or adjuvant-facilitated vaccines or the first part thereof.

In the case of parenteral administration by e.g. subcutaneous injections, the priming treatment comprises at least one administration, preferably 1-15 administrations. Preferably, the initial dose is a low dose corresponding to the initial dose used in conventional parenteral or adjuvant-facilitated vaccines. Suitable dosage ranges can vary within the range from about 0.0001 μg to 1000 μg. Expressed as SQ-u the doses may vary from 20 SQ-u to 100000 SQ-u.

Vaccination will normally be performed from biweekly to bimonthly. This is contemplated to give desired level of prophylactic or therapeutic effect.

In a particular embodiment of the method of the invention, one or more additional rounds of parenteral or adjuvant-facilitated treatment is carried out subsequent to the start of the mucosal SAV to re-stimulate (boost) the immune system further. Such additional rounds of parenteral or adjuvant-facilitated treatment may e.g. involve a limited number of administrations, e.g. from 1-10, preferably 1-5, over a period of e.g. from one to four weeks. Patients may e.g. be subjected to such additional rounds of parenteral or adjuvant-facilitated treatment one or two times each year. Such a treatment protocol has the advantage of being very effective while at the same time limiting the number of parenteral administrations to a minimum. As mentioned above, it is desired to reduce the number of parenteral administrations to a minimum, since such administrations should be performed by specialists and further require post-administration attendance for a period of time.

Adjuvants

Vaccines for parenteral administration may include an adjuvant. Any conventional adjuvant may be used. Suitable adjuvants include oxygen-containing metal salts, microcapsules (microparticles), lipid particles and immunostimulatory molecules.

Examples of suitable oxygen-containing metal salts are e.g. those, wherein the cation is selected from Al, K, Ca, Mg, Zn, Ba, Na, Li, B, Be, Fe, Si, Co, Cu, Ni, Ag, Au, and Cr.

The anion of the oxygen-containing compound may be an organic or inorganic anion, or a combination of organic and inorganic anions. Examples of suitable oxygen-containing metal salts are e.g. those, wherein the anion selected from sulphates, hydroxides, phosphates nitrates, iodates, bromates, carbonates, hydrates, acetates, citrates, oxalates, and tartrates, as well as mixed forms thereof. The oxygen-containing metal salts further comprise coordination complexes. A definition of coordination complexes is given in e.g. The Handbook of Chemistry and Physics 56 Ed., Section B, Chapter 7 (1975-76).

Within the present context, the expression “mixed forms” is intended to include combinations of the various anions as well as combinations with e.g. chlorides, and sulphides.

Although the delivery system comprises an oxygen-containing metal salt, it is contemplated that the oxygen could be substituted by another Group VIA atom such as S, Se or Te.

The oxygen-containing metal salt to be used in accordance with the invention may be any oxygen-containing metal salt providing the desired effect when formulated into a mucosal delivery system. Examples of such oxygen-containing substances are aluminium hydroxide, aluminium phosphate, aluminium sulphate, potassium aluminium sulphate, calcium phosphate, Maalox (mixture of aluminium hydroxide and magnesium hydroxide), beryllium hydroxide, zinc hydroxide, zinc carbonate, zinc chloride and barium sulphate. Preferred oxygen-containing metal salts are aluminium hydroxide, aluminium phosphate and calcium phosphate.

Peyer's patches are aggregates of lymphoid nodules located in the wall of the small intestine, large intestine and appendix and are an important part of body's defense against the adherence and penetration of infection agents and other substances foreign to the body. Peyer's patches are also known as folliculi lymphatic aggregati. Similar folliculi lymphatic aggregati can be found in the respiratory tract, the rectum, the nasal cavity, the oral cavity, the pharynx, the genitourinary tract, large intestine and other mucosal tissues of the body. The said tissues may in general be referred to as mucosally-associated lymphoid tissues (MALT).

It has been shown that pharmaceutically active substances formulated as microcapsules having a proper size and suitable physico-chemical properties may be effectively taken up by Peyer's patches and MALT.

The use of microcapsules involves the advantage of protecting the pharmaceutical active substance from degradation, both during production and storage of the dosage forms, and in the process of administration of the active substance to the patient. This is particularly important, when the active substance is an allergen. The use of microencapsulation to protect sensitive bioactive substances from degradation has become well-known. Typically, a bioactive substance is encapsulated within any of a number of protective wall materials, usually polymeric in nature. The agent to be encapsulated can be coated with a single wall of polymeric material (microcapsules), or can be homogeneously dispersed within a polymeric matrix (microspheres). (Hereafter, the term microcapsules refers to both microcapsules and microspheres and the terms “encapsulation” and “microencapsulation” should be construed accordingly). The amount of substance inside the microcapsule can be varied as desired, ranging from either a small amount to as high as 95% or more of the microcapsule composition. The diameter of the microcapsule is preferably less than 20 μm, more preferably less than 15 μm, more preferably less than 10 μm and most preferably between 1 and 10 μm.

The encapsulating agent may be any biodegradable agent, preferably a polymeric agent. Preferably, the first encapsulating agent is selected from the group consisting of poly-lactide, poly-lactid-poly(ethylene glycol), poly(DL-lactide-co-glycolide), poly(glycolide), copolyoxalates, polycaprolactone, poly(lactide-co-caprolactone), poly(esteramides, polyorthoesters and poly(8-hydroxybutyric acid), and polyanhydrides, most preferably poly(DL-lactide-co-glycolide). Other examples of encapsulating agents are poly(butyl-2-cyanoacrylate), poly(3-hydroxybutyrate) and polyanhydride copolymers of fumaric and sebacic acid, poly(FA:SA). Also, suitable encapsulating agents for use according to the present invention include those derived from animal or vegetable proteins, such as gelatines, dextrins and soy, wheat and psyllium seed proteins; gums such as acacia, guar, agar and xanthan; polysaccarides; starch and modified starch, alignates; carboxymethylcellulose; carrageenans; dextrans; pectins; synthetic polymers such as polyvinylpyrrolidone; and polypeptide/protein or polysaccharide complexes such as gelatine-acacia complexes. In one embodiment of the invention two or more encapsulating agents are used. Preferably, the encapsulating agent is selected so as to make the microparticles hydrophobic. It is believed that hydrophobic microparticles are more easily taken up by the MALT or allowed to elicit its effects via the MALT.

Lipid adjuvants include oil-in-water emulsions, liposomes, virosomes, ISCOM's and cochleates.

Examples of oil-in water emulsions are MF59, which is a squalene in water emulsion, Freund's adjuvant and SAF.

Liposomes are aqueous suspensions of spheroid vesicles, which are phospholipids organised in bilayer structures. Liposomes are generally composed of phospholipids and cholesterol. Any phospholipids may be used for the preparation of liposome vaccines. One example of a suitable phopholipid is dipalmitoyl phophatidylcholine. One example of a liposome vaccine composition is dipalmitoyl phophatidylcholine, cholesterol, diacetylphophate and antigen. Liposomes are classified according to size and properties as follows: Small unilamellar vesicles (SUV), Large unilamellar vesicles (LUV), LUV/reverse phase evaporation (REV), Large unilamellar vesicles by extrusion (LUVET), multilamellar vesicles (MLV), freeze and thaw multilamellar vesicles (FT-MLV), stable pluerilamellar vesicles (SPLV).

Saponins are the active component of a variety of lipid mixtures known as ISCOMs (Immunostimulating complexes). Saponins are sterol and triterpenoid glycosides derived from the bark of the Quilaja saponiaria tree. Examples of ISCOMs are Quil A and Qs-21.

Immunostimulatory molecules include cytokines, including IL-2, IL-12, GM-CSF), MDF derivatives, CpG oligonucleotides, LPS, MPL and phosphosphazenes.

The adjuvant in the adjuvant-facilitated mucosal vaccine may be any conventional adjuvant, including heat-labile enterotoxin (LT), cholera toxin (CT), cholera toxin B (CTB), polymerised liposomes, mutant toxins, e.g. LTK63 and LTR72, and microcapsules.

Detection of Activation of the Systemic Immune System

A particular embodiment of the invention comprises the further step of performing a measurement of an activation marker to determine, whether the activation of the systemic immune system has been effected.

The activation of the systemic immune system may be detected by any suitable activation marker of the systemic immune system, including antibodies and markers for effector cell activation.

In a particular embodiment of the invention, the marker for the activation of the systemic immune system is one or more antibodies specific to the antigen in question, including IgA, IgD, IgE, IgG, IgM and any subclass thereof. The detection of antibodies may be carried out using any class, sub-class or combination thereof, including IgA, IgA1, IgA2, IgD, IgE, IgG, IgG1, IgG2, IgG3, IgG4, IgM.

In a preferred embodiment of the invention the activation marker is IgG specific to the antigenic agent. In this connection, “IgG” means total IgG or IgG1, IgG2, IgG2a, IgG2b, IgG3, IgG4 or any subtotal thereof. In a further preferred embodiment the activation marker is IgG₄ specific to the antigenic agent.

The detection of antibodies specific for the mucosal antigenic agent may be carried out using any conventional immunoassay. Preferred immunoassays are those mentioned in WO 94/11734 and WO 99/67642.

In a further preferred embodiment of the invention the activation marker is IgX. In a preferred embodiment of the invention, the IgX is expressed as the ratio of the level of IgE as measured in an immunoassay with competition from other components of the biological sample to the level of IgE as measured in an immunoassay with no competition.

In another particular embodiment of the method of the invention, the activation of the systemic immune system is measured as the ability to activate effector cells of the immune system.

In one embodiment of the invention, whole blood is used for effector cell activation. In a second embodiment, the effector cells are cells isolated from a biological sample. In a third embodiment, the effector cells are cells isolated from a biological sample and cultivated. In a fourth embodiment, the effector cells are cells isolated from a biological sample, cultivated and modified, e.g. genetically modified.

Preferably, the effector cells are selected from the group consisting of mast cells, basophils, eosinophils, T cells, B cells and Antigen Presenting Cells (APC), and combinations thereof. Other preferred effector cells are modified effector cells, i.e. cells derived from and having at least some features of effector cells, including genetically modified cells and malignantly transformed cells.

In one embodiment of the invention, the effector cell activating ability is measured by measuring the level of an effector cell marker. The marker is preferably selected from the group consisting of secretory molecules, surface molecules and intracellular molecules. Preferably, the secretory molecule is selected from the group consisting of mediators, cytokines, cytotoxic proteins and soluble receptors.

Examples of the mediator to be measured are mediators selected from the group consisting of histamine, leucotrienes (LTB₄, LTC₄, LTD₄ and LTE₄), prostaglandines (PGD₂, PGE₂ and PGF₂ₐ), thromboxane, Platelet Activating Factor (PAF), Major Basic Protein (MBP), ECF, ECP, EDN, EPO, bradykinin, adenosine, Substance P, Neurokinin A, complement factors (e.g. C3d), including complement fragments; Serotonin, Oxygen Radicals, basogranulin, and mast cell and basophil proteases, including tryptase, chymase, carboxypeptidase and cathepsin.

Examples of the cytokine to be measured are cytokines selected from the group consisting of Interieukins (IL-1 to IL-27), hematopoietric growth factors, granulocyte-macrophage colony stimulating factors (e.g. CM-CSF), interferons (IFNα, IFNβ, IFNγ), tumor necrosis factor (TNF) related molecules (TNF and lymphotoxin), Ig superfamily members (IL-1), the TGF-beta family and the chemokines (IL-8, RANTES and others). Assays for measuring the following cytokines are widespread: IL-2, IL-4, IL-5, IL-6, IL-10, IL-12, IL-13, IFN-gamma, TNF-alpha, TGF-beta.

Examples of the cytotoxic protein to be measured are cytotoxic proteins selected from the group consisting of Eosinophil Cationic Protein (ECP), Major Basic Protein (MBP) and EDN.

Preferably, the surface molecule is selected from the group consisting of surface receptors and adhesion molecules, such as selectins, integrins and the immunoglobulin superfamily (ICAM-1, VCAM-1), VLA4, CD11B, CD11C, CD18 and a-d. Surface molecules known to be up- or downregulated in effector cells by antigen activation are CD23, CD69, CD203C (1-NPP3), CD31, CD162 and CD162L. Other surface molecules include basogranulin.

Preferably, the effector cell marker is histamine, tryptase, basogranulin, leucotrien LTC₄, CD63, CD69 and CD203C. Histamine may e.g. be measured in an ELISA-based method based on the competition between histamine to be assayed and its enzyme conjugate, histamine-alkaline phosphatase used as tracer for binding to antibody coated onto microwells. The monoamine histamine is too small to occupy completely the binding site on the antibody. High affinity monoclonal antibodies directed against modified histamine have therefore been obtained. The histamine in the sample must be derivatized in the same manner as the histamine of the conjugate. This is achieved readily and reproducibly with an acylating reagent at slightly alkaline pH. The acylated histamine of the sample, and the histamine-alkaline phosphatase conjugate, when added to the microtiter wells, compete for binding to a limiting number of antibody sites. After incubation, the wells are rinsed in order to remove non-bound components. The bound enzymatic activity is then measured by the addition of a chromogenic substrate (pNPP). The intensity of the color depends inversely on the concentration of histamine in the sample. The concentration is calculated on the basis of a standard curve obtained with standards. This enzyme immunoassay may be carried out using a kit obtainable from “IMMUNOTECH” (Marseille, France).

In a second embodiment of the invention, the effector cell activating ability is measured by measuring the T cell proliferation. The T cell proliferation may be measured by a method based on incorporation of 3H-thymidine or the reduction of fluorescence labelling and may be conducted using freshly isolated leucocytes from the blood of sensitized subjects or using established allergen-specific T-cell lines. In addition, the cytokine production of the activated cells may be investigated by analysis of the cell supernatants by ELISA or beads based methods.

Early events in the T-cell activation may be investigated through flow cytometric analysis of T-cell expression of different surface receptors such as CD25, 26, 27, 39, 45 RA/O, 69, 70, 96, 97, 108, 109, 134 (OX40), 153, 154 (OX40L), 166, 178 (FasL), 183 (CXCR3), 212 (IL-12Rb1), 223, which are up- or down-regulated at different time-points during T-cell activation.

The activation of T-cells may be influenced by the differentiation and activation stage of the antigen presenting cells (APC), which may be investigated by flow cytometric analysis of the following surface molecules: CD14, 25, 26, 40, 80/86, 83, 105, 166.

Finally, the activation of the systemic immune system by the priming treatment may be detected as B-cell activation, which may be described via the surface expression of CD25, 26, 39, 80/86, 97, 126, 138 and surface expression as well as secretion of different antibody isotypes. As an additional alternative the majority of the parameters described above may be investigated at the mRNA level through Taqman analysis, gene chip analysis, or other method for quantifying gene expression.

The biological sample to be assayed for the presence of one or more antibodies specific to the antigenic agent in question may be any biological sample, which potentially contains the activation marker, including any biological fluid or mixture, which is excreted, secreted or transported within a biological organism. The biological sample may e.g. be blood, plasma, serum, urine or saliva.

An activation of the systemic immune system is considered to have been made, when a significant change in the level of the activation marker compared to the level at the start of the priming treatment has been effected. What constitutes a significant change depends strongly on the marker in question, but this will be common technical knowledge for a person skilled in the art or he will be able to establish this through appropriate tests.

For the activation marker IgX, the significant change is preferably a reduction of more than 10%, more preferably more than 20% and most preferably more than 30%. For the activation marker specific IgG, IgG1, IgG2, IgG3, IgG4, the significant change is preferably an increase of more than 10%, more preferably more than 20% and most preferably more than 30%.

Specific Allergy Vaccination (SAV) by Means of Non-Gastrointestinal Mucosal Administration

Specific allergy vaccination (SAV), formerly known as Specific Immunotheraphy or Hyposensitization, has been used for the treatment of Type 1 IgE mediated allergic disease since the beginning of this century.

The general benefits obtained through SAV are: a) reduction of allergic symptoms and medicine consumption, b) improved tolerance towards the allergens in the eyes, nose and lungs and c) reduced skin reactivity (early and late phase reactions).

The basic mechanism behind the improvement obtained by SAV is unknown, but a number of common features can be extracted from the numerous SAV studies performed in the last decades: 1) the amount of total IgE is unchanged during the treatment period, 2) the amount of allergen specific IgE increases transiently during updosing, then it falls back to the initial (pretreatment) level, 3) the epitope specificity and affinity of IgE remains unchanged, 4) allergen specific IgG, in particularly IgG4, raises sharply during SAV, 5) a new Th0/1 response is apparently initiated and 6) the Th2 response seem unchanged. There is no correlation between the effect induced by SAV and the onset of specific IgG.

SAV induces a new immune response which matures during the treatment period (Th0/1 T-cells are recruited, an allergen specific IgX (X may be Al, A2, G1, G2, G3, G4, M or D) is initiated). As the affinity (or amount/affinity) of the new antibody response, IgX, has matured, IgX may compete efficiently with IgE for the allergen(s), inhibiting the “normal” Th2 based allergic response characterised by the cross-linking of receptor bound IgE on the surface of mast-cells and basophils. Hence, clinical symptoms will gradually be reduced.

The SAV carried out in accordance with the present invention requires that the subject are pre-sensitised with the allergen in question by a priming treatment. The object of the SAV, which is carried out by non-gastrointestinal mucosal administration, is to elicit an allergen specific antibody response in the mucosa of the subject, the antibody response comprising at least an IgA and/or an IgM response. However, the antibody response elicited may comprise other allergen specific antibodies, including IgD, IgE, IgG, IgD and any subclass thereof. In accordance with the SAV of the invention, the antibody response is elicited in all mucosa of the subject, including the mucosa of the respiratory system, the mucosa of the digestive system, the rectal mucosa and the genital mucosa.

The SAV by means of non-gastrointestinal mucosal administration includes nasal, vaginal, sublingual, ocular, rectal, urinal, intramammal, pulmonal, otolar (i.e. via the ear), buccal administration or oromucosal administration. The mucosal vaccine may be in the form of a spray, an aerosol, a mixture, a suspension, a dispersion, an emulsion, a gel, a paste, a syrup, a cream, an ointment, implants (ear, eye, skin, nose, rectal, and vaginal), intramammary preparations, vagitories, suppositories, or uteritories.

It has been speculated that it is preferable to carry out a mucosal administration of a vaccine via the mucosa, which is subject to the natural exposure to the antigenic agent. Accordingly, for infections and allergies caused by airborne mucosal antigenic agents, it is preferred to use administration via the respiratory system, preferably an oromucosal administration.

The non-gastrointestinal mucosal vaccine may include an adjuvant, which may be any conventional adjuvant, including heat-labile enterotoxin (LT), cholera toxin (CT), cholera toxin B (CTB), polymerised liposomes, mutant toxins, e.g. LTK63 and LTR72, and microcapsules.

In one embodiment of the invention, the subject is subjected to a vaccination protocol comprising daily administration of the vaccine. In another embodiment of the invention the vaccination protocol comprises administration of the vaccine every second day, every third day or every fourth day. For instance, the vaccination protocol comprises administration of the vaccine for a period of more than 4 weeks, preferably more than 8 weeks, more preferably more than 12 weeks, more preferably more than 16 weeks, more preferably more than 20 weeks, more preferably more than 24 weeks, more preferably more than 30 and most preferably more than 36 weeks.

The period of administration may a continuous period. Alternatively, the period of administration is a discontinuous period interrupted by one or more periods of non-administration. Preferably, the (total) period of non-administration is shorter than the (total) period of administration.

In a further embodiment of the invention, the vaccine is administered to the test individual once a day. Alternatively, the vaccine is administered to the test individual twice a day. The vaccine may be a uni-dose vaccine.

Preferably, in the mucosal SAV in the method according to the present invention, doses corresponding to those used in conventional mucosal SAV without priming treatment are used.

Oromucosal Administration

The oromucosal administration may be carried out using any available oromucosal administration formulation, including a solution, a suspension, fast dispersing dosage forms, drops and lozenges.

In a preferred embodiment of the invention, sublingual immunotherapy (SLIT) is used, in which case fast dispersing dosage forms, drops and lozenges are preferred formulations.

Examples of fast dispersing dosage forms are those disclosed in U.S. Pat. No. 5,648,093, WO 00/51568, WO 02/13858, WO99/21579, WO 00/44351, U.S. Pat. No. 4,371,516 and EP-278 877, as well as co-pending DK PA 2003 00279 and DK PA 2003 00318 filed in the assignee name of ALK-Abelló A/S. Preferred fast dispersing dosage forms are those produced by freeze-drying. Preferred matrix forming agents are fish gelatine and modified starch.

Classical incremental dosage desensitisation, where the dose of allergen in the form of a fast dispersing solid dosage form is increased to a certain maximum relieves the symptoms of allergy. The preferred potency of a unit dose of the dosage form is from 150-1000000 SQ-u/dosage form, more preferred the potency is from 500-500000 SQ-u/dosage form and more preferably the potency is from 1000-250000 SQ-u/dosage form, even more preferred 1500-125000 SQ-u/dosage form most preferable 1500-75000 SQ-u/dosage form.

In another embodiment of the invention the dosage form is a repeated mono-dose, preferably within the range of 1500-75000 SQ-u/dosage form.

Detection of Elicitation of Ig Response in the Mucosa

A particular embodiment of the invention comprises the further step of performing a measurement of a mucosal marker to determine, whether an antigenic agent specific IgA and/or IgM antibody response in the mucosa of the subject has been elicited. Preferably, the IgA and/or IgM antibody response increases at least 5%, preferably 10%, more preferably 50%, more preferably 100% and most preferably 200% compared to the level before the priming treatment.

When the mucosal antigenic agent is airborne and hence comes into contact with the mucosa of the respiratory system, it is preferably determined whether an antigenic agent specific Ig antibody response in the respiratory system mucosa of the subject has been elicited.

The level of IgA and/or IgM in the mucosa of the respiratory system may e.g. be measured in a biological sample in the form of broncho-alveolar lavage (BAL) or nasal lavage (NAL).

When the mucosal antigenic agent is a food antigenic agent and hence comes into contact with the mucosa of the digestive system, it is preferably determined whether an antigenic agent specific Ig antibody response in the digestive system mucosa of the subject has been elicited.

Method of Evaluating the Immunological Status of a Subject

The present invention further relates to a method of evaluating the immunological status of a subject comprising
a) measuring the level of mucosal Ig specific for an antigenic agent, and
b) using the measurement to evaluate the immunological status of the subject.

This aspect of the invention is based on the recognition that the level of mucosal IgA and IgM against a specific antigenic agent holds information about the immunological status of the subject, in particular about the ability of the subject to respond to exposure to the antigenic agent. The evaluation may e.g. be based on a comparison of the measured level with levels of IgA and IgM specific for the same antigenic agent or another antigenic agent of subjects, with known immunological status.

In a preferred embodiment of this method of the invention, the measurement of mucosal IgA and/or IgM is used to evaluate the effect of the method of preventing or treating an allergy to or an infection by a mucosal antigenic agent in a subject, i.e. the method of claim 1.

Preferably, the level of mucosal IgA and/or IgM is measured two or more times during the course of the method of claim 1, and the change in the measured level is used to evaluate the effect of the method.

Second Method of Preventing or Treating an Allergy to or an Infection by a Mucosal Antigenic Agent

The present invention also relates to a second method of preventing or treating an allergy to or an infection by a mucosal antigenic agent in a subject comprising
a) subjecting the subject to a priming treatment by means of oromucosal administration of the antigenic agent to activate the mucosal immune system, and
b) subjecting the subject to Specific Allergy Vaccination (SAV) by means of parenteral or adjuvant-facilitated mucosal administration of the antigenic agent to elicit an antigenic agent specific Ig antibody response in the respiratory system mucosa of the subject.

The second method of the invention is based on the same general concept and inventive rationale as the method of claim 1 of the present invention. Accordingly, the two variants of the method of preventing or treating an allergy to or an infection by a mucosal antigenic agent in a subject both relate to a method comprising two steps using different administration routes, viz. parenteral or adjuvant-facilitated mucosal on the one hand and mucosal on the other hand, the difference between the two variants being the sequence of the two steps. The present invention is further based on the surprising finding that the same advantages and effects may be obtained regardless of the sequence of the steps used.

Accordingly, the invention is based on the surprising finding that it is possible to elicit a strong mucosal IgA and IgM immune response by means of oromucosal administration to prime the immune system in combination with subsequent parenteral administration, and that such a vaccination protocol is an effective treatment of allergies and infections, wherein the action of the antigenic agent is effected via contact with the mucosa. It is believed that the mechanism of action of this treatment is that the IgA and IgM bind to the antigenic agent thereby neutralising it. The invention is further based on the surprising finding that carrying out the priming treatment step of such a combination treatment by means of oromucosal administration is more effective than other types of mucosal treatment.

As mentioned above, conventional vaccination is not convenient due to the need for a vaccination programme involving many injections over an extended period of time. A further advantage of the method of the present invention is that compared to conventional parenteral vaccination, the number of required parenteral administrations is reduced significantly. For example, the method of the invention has provided a possibility to conduct the up-dosing phase of the vaccination programme, and even the first few administrations of the maintenance phase, by means of oromucosal administration, and then to conduct only the maintenance phase, or the rest of the maintenance phase, respectively, by means of parenteral administration.

In the method of the invention, the oromucosal priming treatment may be carried out using the same doses and frequency of administration as used in conventional mucosal SAV with no subsequent parenteral or adjuvant-facilitated treatment, or as set out in the above sections concerning “Specific Allergy Vaccination (SAV) by means of mucosal administration” and “Oromucosal administration”. The duration of the priming treatment is at least one day, preferably 1-50 day, more preferably 3-30 days and most preferably 5 to 20 days.

The oromucosal priming treatment is followed by parenteral or adjuvant-facilitated SAV, which may be carried out with the same duration and frequency as conventional parenteral or adjuvant-facilitated SAV with no preceding mucosal priming treatment, or as set out in the above section “Priming treatment”. The doses may e.g. be the same as the maintenance dose for conventional parenteral or adjuvant-facilitated SAV with no preceding oromucosal priming treatment, or the dose may be reduced compared to the said maintenance dose, e.g. so as to use from 10 to 100% of the maintenance dose. Also, the parenteral or adjuvant-facilitated SAV of the present method may involve a full or partly up-dosing, which may e.g. be carried out in the same manner as in connection with conventional parenteral or adjuvant-facilitated SAV with no preceding oromucosal priming treatment.

In a particular embodiment of the method of the invention, one or more additional rounds of treatment by mucosal administration is carried out subsequent to the start of the parenteral or adjuvant-facilitated SAV to re-stimulate (boost) the immune system further. Such additional rounds of mucosal treatment may e.g. involve a treatment protocol corresponding to that of the priming treatment or a part of the priming treatment. Patients may e.g. be subjected to such additional rounds of mucosal treatment one or two times each year. Such a treatment protocol has the advantage of being very effective while at the same time limiting the number of parenteral administrations to a minimum. As mentioned above, it is desired to reduce the number of parenteral administrations to a minimum, since such administrations should be performed by specialists and further require post-administration attendance for a period of time.

A typical progression course of allergy in a subject may start with sensitisation against one allergen, then sensitisation against other allergens, then symptoms of an allergy towards one allergen, then worsening of those symptoms and finally symptoms of allergies towards other allergens. It is theorised that both the prevention and treatment of allergy according to the present invention is in general more effective the less progressed the allergy is.

Accordingly, in a particular embodiment of the invention directed to the prevention or treatment of allergy, the subject does not exhibit symptoms of the allergy for which treatment is given. In another particular embodiment, the subject does not exhibit any symptoms of allergy. In a further particular embodiment of the invention the subject is not sensitised to the allergy for which treatment is given. In yet a further embodiment of the invention the subject is not sensitised to any allergy.

Method of Screening Mutants of Antigens

The present invention further relates to a method of screening a mutated antigenic agent for its ability to elicit an immune response comprising
a) subjecting a subject to a priming treatment using an allergen corresponding to the wild type allergen of the mutated allergen to activate the immune system,
b) subjecting the subject to Specific Allergy Vaccination (SAV) using the mutated allergen, and
c) examining whether the mutated allergen elicits an immune response.

This aspect of the invention like the other aspects is based on the experimental finding that the effect of Specific Allergy Vaccination (SAV) is strongly increased, when a subject has been subjected to a priming/sensitising exposure to the antigenic agent. This aspect of the invention is further based on the recognition that this fact may be used in a method for screening a mutated antigenic agent for its ability of eliciting an immune response by subjecting a subject primed with the wild-type antigenic agent to the mutated agent and examining whether it elicits a strong immune response similar to that expected of the wild-type agent, i.e. whether the mutated antigenic agent immunologically resembles the wild-type agent sufficiently to be capable of using the priming effect of the wild-type agent.

In this aspect of the invention the priming treatment may be carried out by any known administration route, including parenteral and mucosal administration. Likewise, the SAV using the mutated allergen may be carried out using any known administration route, including parenteral and mucosal administration.

The examination of whether the mutated allergen elicits an immune response may be carried out by measuring a marker of the relevant compartment of the immune system in respect to the administration routes used in the priming and subsequent SAV treatments. For example, the examination of whether the mutated allergen elicits an immune response may be carried out by measuring a marker of the mucosal immune system, e.g. IgA and IgM, or by measuring a marker of the systemic immune system.

Definitions

The expression “allergy” means any type 1, 2, 3 or 4 hyper-sensitivity allergy.

The expression “infection” means an infection by a mucosal antigenic agent.

The expression “mucosal antigenic agent” means any antigenic agent which comes into contact with the mucosa of the subject.

The term “mucosal administration” means administration via one or more immunocompetent mucosa.

The term “adjuvant-facilitated mucosal administration” means mucosal administration using an adjuvant.

The expression “digestive antigenic agent” means any antigenic agent which comes into contact with the mucosa of the digestive system, in particular the mucosa of the oral cavity, the pharynx, the larynx, the stomach and the intestine.

The expression “respiratory antigenic agent” means any antigenic agent which comes into contact with the mucosa of the respiratory system, in particular the mucosa of the nose, the oral cavity, the pharynx, the larynx, the trachea and the lungs.

The term “oromucosal” means relating to the mucosa of the oral cavity, the mucosa of the pharynx and the sublingual mucosa.

The term “buccal” means relating to the oral cavity.

The term “sublingual” means relating to the position below the lingua of the oral cavity.

The expression “oromucosal administration” means administration via the buccal, the sublingual and/or the pharynx mucosa.

The expression “non-gastrointestinal mucosal administration” means administration via any immuno-competent mucosa of the body except the mucosa of the stomach and intestines, including i.a. the nasal, vaginal, sublingual, ocular, rectal, urinal, intramammal, pulmonal, otolar (i.e. via the ear), buccal and oromucosal mucosa.

The expression “allergen” means any allergen, which comes into contact with the mucosa of the subject, and it refers to any naturally occurring protein or mixture of proteins that have been reported to induce allergic, i.e. IgE mediated reactions, upon repeated exposure to an individual. The allergen may be used in the form of an allergen extract, a purified allergen, a modified allergen, a recombinant allergen, a recombinant mutant allergen, any allergen fragment above 30 amino acids or any combination thereof.

The term “mucosal Ig” means any immunoglobulin present in the mucosa, including IgA and IgM.

The term “systemic immune system” means the immune system of the whole organism as opposed to a compartmental immune system of a tissue or a set of tissues, such as the immune system of the mucosa, of the body cavities (peritoneum or pleura) or the skin.

The term “fast dispersing dosage form” refers to dosage forms which disintegrate in less than about 90 seconds, preferably in less than 60 seconds, preferably in less than 30 seconds, more preferably in less than 20, even more preferably in less than 10 seconds in the oral cavity, even more preferred in less than 5, most preferably in less than about 2 seconds of being placed in the oral cavity.

The term “non-compressed” refers to a solid dosage form, which is manufactured by removal of a liquid from a solidified system comprising matrix forming agents, active ingredient and other suitable ingredients resulting in a an allergen comprised solid matrix.

The term “solid dosage form” refers to a dosage form, which is not a liquid, nor a powder, e.g. a tablet or a granular composition, when it is administered in the oral cavity.

The term “SQ-u” means SQ-Unit: The SQ-Unit is determined in accordance with ALK-Abelló A/S's “SQ biopotency”-standardisation method, where 100,000 SQ units equal the standard subcutaneous maintenance dose.

Normally 1 mg of extract contains between 100,000 and 1,000,000 SQ-Units, depending on the allergen source from which they originate and the manufacturing process used. The precise allergen amount can be determined by means of immunoassay i.e. total major allergen content and total allergen activity.

The term “IgX” means a parameter expressing directly or indirectly the level of allergen specific non-IgE antibodies, such as IgG4, present in the biological sample, which can compete with IgE on the binding of the allergen.

IgX may e.g. be the absolute IgX level or the ratio of IgE as measured in an immunoassay with competition (interference) from other components of the biological sample to the level of IgE as measured in an immunoassay with no competition.

### EXAMPLES

### Example 1

### Two-Stage Treatment Comprising Priming by Parenteral Administration and SAV by Sublingual Administration in Mice

Introduction

To understand the immunoregulatory mechanisms induced by Sublingual Immunotherapy (SLIT), a study of a treatment method according to the present invention comprising sublingual allergen administration in primed mice was carried out.

The present study demonstrates substantial changes in the mucosal IgA response upon SLIT treatment with an extract of Phleum pratense (Phl p).

The mice were primed by three intraperitoneal (ip) injections of aluminiumhydroxide-adsorped Phl p followed by SLIT administration as outlined above Blood samples were taken at relevant time points and washes of the lungs (BAL) as well as the nasal passages (NAL) were collected at time of sacrifice. The spleens were collected for in vitro analysis of Phl p specific proliferation and cytokine secretion. Serum samples were analyzed for Phl p specific IgE, IgG1, IgG2a and total IgG. Specific IgA was measured in BAL and NAL. Spleens were collected for T-cell analysis.

Methods

Animals

Female, 6-10 week-old BALB/c mice were bred in-house and maintained on a defined diet not containing components cross reacting with antisera to Phl p. Each experimental group consisted of 8-10 animals.

Animal Experiments

The experimental design was as follows: Week 0 was defined as the start of the experiment. The mice were primed with i.p. injections (5 kSQ/alum) at weeks 0, 2 and 4, and then the mice were divided into four groups receiving SLIT treatment by buccal administration of drops (Phl p extract dissolved in buffer) with doses 0 (buffer only), 5, 25 and 125 kSQ for 2, 4 and 6 weeks (five times per week), after which the mice were sacrificed. Serum samples for mice having received six weeks of SLIT treatment were analyzed for Phl p specific IgE, IgG1, IgG2a and total IgG. BAL and NAL fluid was collected at the time of sacrifice. Specific IgA was measured in BAL and NAL for mice having received 2, 4 and 6 weeks of SLIT. The spleens were collected for in vitro analysis of Phl p specific T-cell proliferation and cytokine secretion.

IgA Assay

Estapore magnetic beads (Estapore IB-MR/0.86) coupled to goat a-mouse IgA are incubated with BAL or NAL. Then washing and incubation with biotinylated allergen is carried out. Then washing and incubation with streptavidin labeled LITE reagent is carried out, and after washing light luminescence is measured in a luminometer (Magic Lite Analyser EQ).

IgE Assay

Estapore magnetic beads (Estapore IB-MR/0.86) coupled to a-mouse IgE A0201 are incubated with mouse serum. Then washing and incubation with biotinylated allergen is carried out. Then washing and incubation with streptavidin labeled LITE reagent is carried out, and after washing light luminescence is measured in a luminometer (Magic Lite Analyser EQ).

IgG, IgG1 and IgG2a Assay

1. Coating. 100 μl Phl p (10 μg/ml) extract is added to the wells of an ELISA plate (NUNC Maxisorp 439454). The plates are allowed to stand until the next day at 4-8° C.
2. Washing. The coated plates are washed with a buffer.
3. Blocking. 200 μl 2% Casein buffer is added to each well and incubated at room temperature for one hour on a shaking table. After incubation the Casein buffer is removed.
4. Serum. The serum sample is diluted, and 100 μl diluted sample is added to the well of a plate and incubated at room temperature for two hours on a shaking table.
5. Washing.
6. Conjugate. 100 μl biotinylated rabbit anti-mouse IgG/IgG1/IgG2a diluted in 0.5% BSA buffer is added to each well and allowed to stand at room temperature for one hour on a shaking table.
7. Washing.
8. 100 μl Streptavidin-HRP diluted in 0.5% BSA buffer is added to each well and allowed to stand at room temperature for one hour on a shaking table.
9. Substrate: 100 μl TMP (3,3′,5,5′-Tetramethylbenzidine, Kem-En-Tec TMB ONE) is added to each well and incubated 20 min.
10. Stop. 100 μl 0.5 M H₂SO₄ is added to each well to stop the reaction.
11. Measurement. The resulting reaction mixtures are subjected to a spectrophotometric measurement at 450 nm endpoint (Bio Kinetics Reader EL-340).
   T-Cell Proliferation Assay

Spleens were teased into single cell suspension and washed three times in medium. Cells were counted and adjusted to 1.67×10⁶ cells/mL. 3×10⁵ cells were added to each well of a 96 well flat-bottomed culture plate and the cells were stimulated by 0, 10 and 40 μg/mL Phleum pratense extract. The cells were cultured for 6 days at 37° C. and 5% CO₂. Proliferation was measured by adding 0.5 μCi of ³H-thymidine to each well for the last 18 hours of the culture period, followed by harvesting the cells and counting the incorporated radiolabel.

Cytokine Measurements

Spleens were teased into single cell suspension and washed three times in medium. Cells were counted and adjusted to 3×10⁶ cells/mL. 2.5×10⁶ cells were added to each well of a 24 well culture plate and the cells were stimulated by 0 and 40 μg/mL Phleum pratense extract. Supernatants, harvested at day 3 and day 6, were analyzed for the presence of IL-2, IL-4, IL-5, Interferon gamma and Tumor necrosis factor alpha using the cytometric bead array assay from Becton Dickinson. In brief, the above mentioned supernatants were mixed with fluorescent beads coated with cytokine specific capture antibodies as well as PE-conjugated, cytokine specific detection antibodies. After washing unbound material away the sample data were acquired using a flow cytometer.

Assay Measuring the Ability of Mucosal IgA to Block IgA Binding to Allergen

This assay is carried out by first incubating serum with anti-mouse IgE coupled paramagnetic particles. After washing the particles, both IgA containing BAL and biotinylated Phl p extract is added to the particles for overnight incubation. Streptavidin conjugated to acridinium ester is added to detect bound Phl p extract. Visualisation is carried out in a luminometer with automated injection of peroxide and hydrochloric acid. The read-out is RLU (Relative Light Units)

Results

Antibody Response

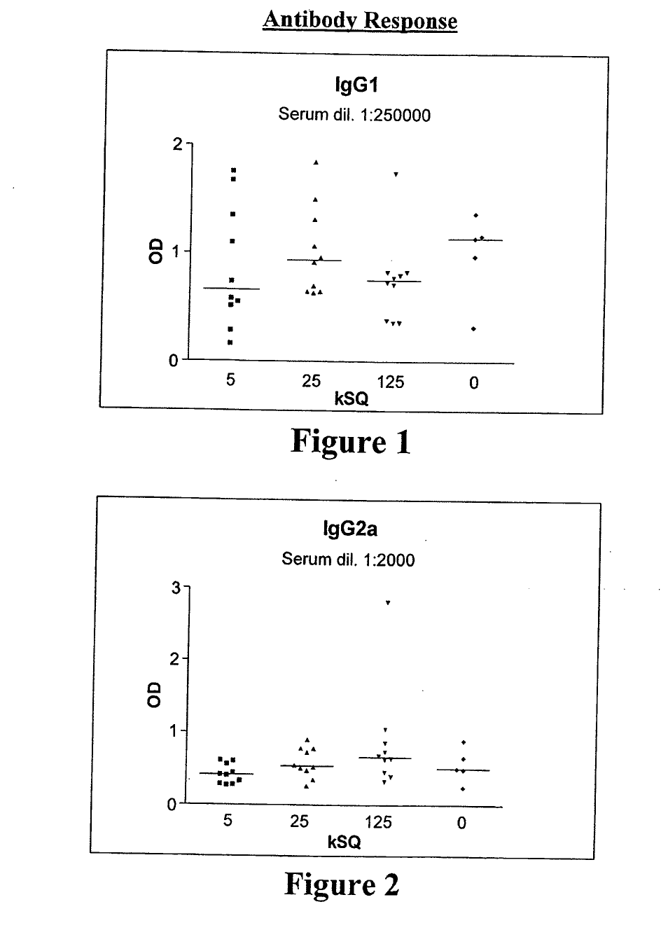

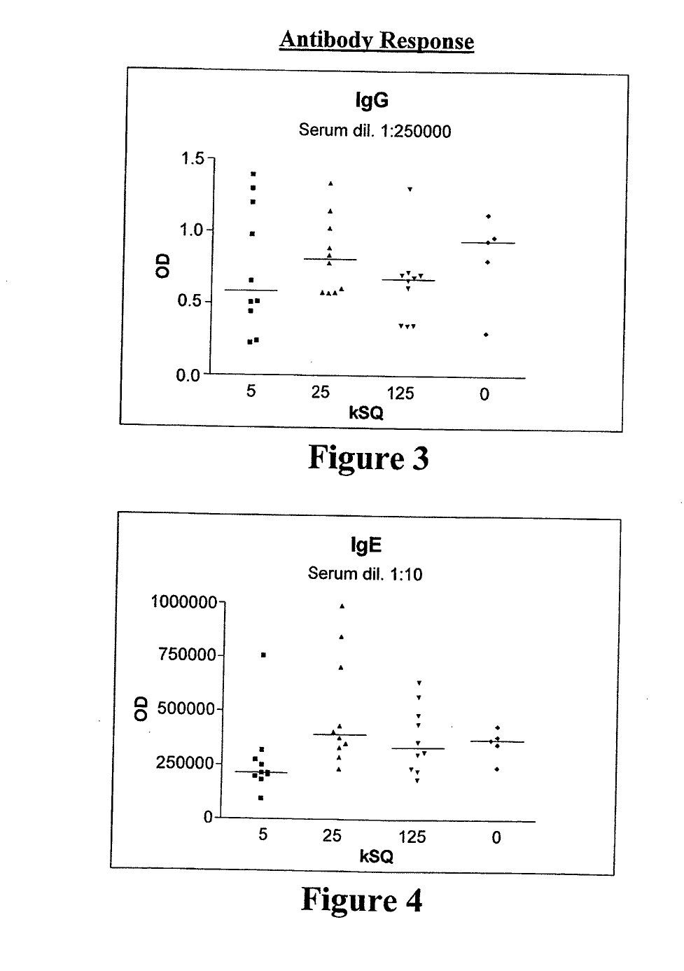

The results are shown in FIGS. 1-6 showing IgG₁, IgG₂, total IgG, IgE, IgA (BAL) and IgA (NAL), respectively.

SLIT administration in mice primed by three i.p. immunisations, does not generate increased serum levels of IgE and IgGs (FIG. 1-4). Also, no down-regulation of the antibody response is observed, suggesting that SLIT in previously primed mice does not induce active suppression of the immune response.

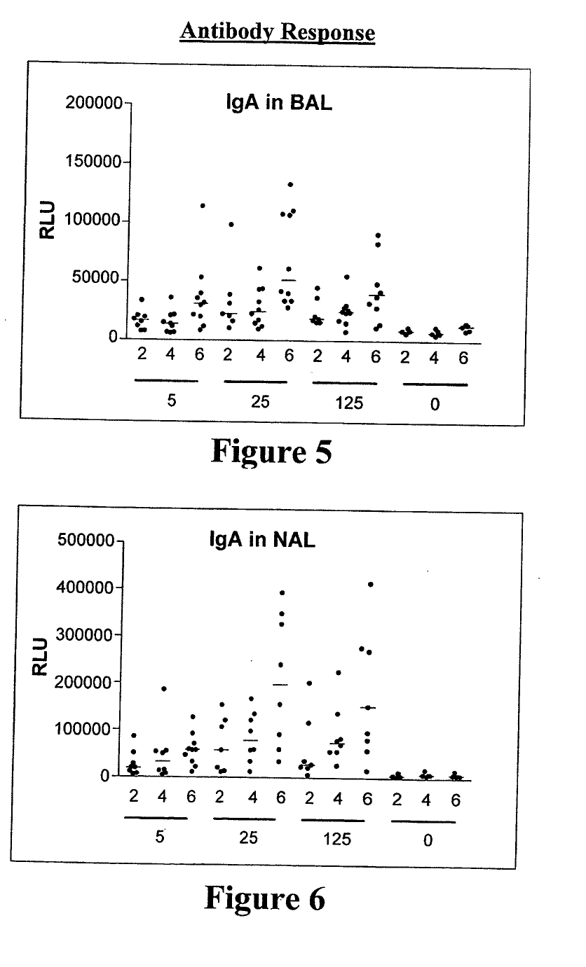

The absence of modulation in the IgG and IgE antibody responses is not reflected in the mucosal IgA response. The levels of IgA in BAL and NAL are from 5 to 30 times higher in Phl p-SLIT treated mice vs. buffer treated mice. The IgA levels are proportional to the time length of SLIT administration as well as to the dose of administration. However, there seems to be a tendency towards a plateau or maximum at the intermediate dose of 25 kSQ and treatment for 6 weeks (FIGS. 5 and 6). The BAL and NAL fluid were also analysed for the presence of Phl p specific IgE antibodies. As opposed to murine asthma disease models, we do not find IgE antibodies in BAL or NAL, probably due to lack of pulmonary inflammation in the SLIT-treated mice (data not shown).

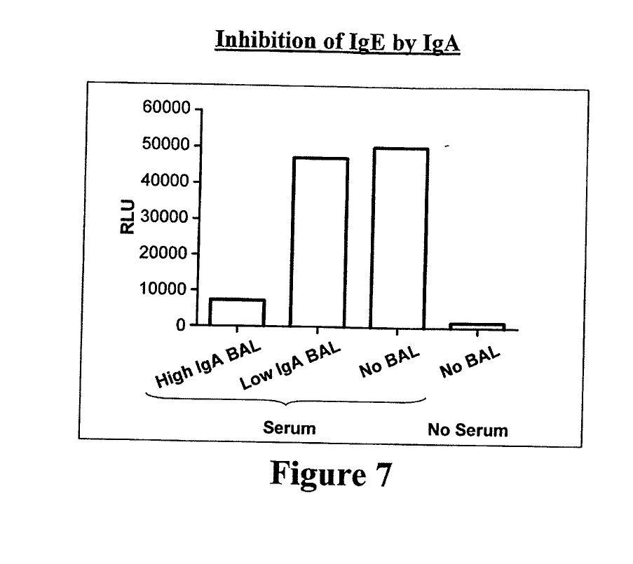

Next it was investigated whether the secreted, mucosal IgA from BAL is able to block the binding of serum IgE to a biotinylated extract of Phl p. As shown in FIG. 7, mucosal IgA is able to inhibit the binding of IgE to biotinylated Phl p extract, indicating identical recognition sites or epitopes for IgE and mucosal IgA. The columns No serum-No BAL and Serum-No BAL designates a negative and a positive control, respectively. The two left hand columns show inhibition of Phl p binding to IgE by BAL fluids containing high and low levels of IgA, respectively. The mucosal IgA response increases with the duration of the SLIT treatment. The mucosal IgA response also increases with the dose, although a plateau appears at the 25 kSQ dose when treating for six weeks.

T-Cell Response

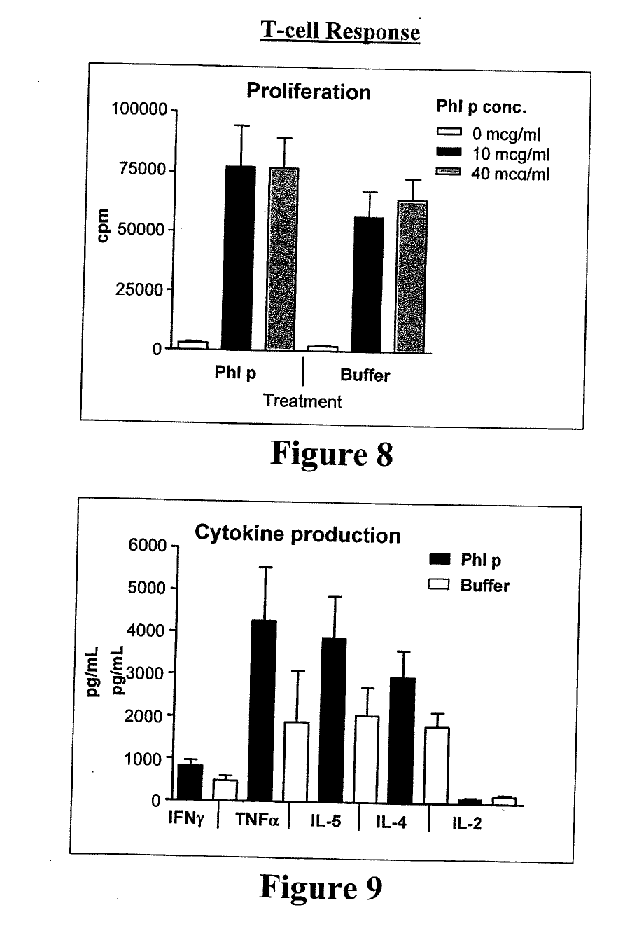

When mice were sensitized by three ip injections of Phl p in alum prior to treatment with Phl p SLIT, no difference between Phl p treated mice and buffer treated mice was observed. As depicted in FIG. 8, there is a tendency towards a slightly increased proliferative response in mice treated with Phl p SLIT, but this was not significant. With regards to cytokine production (FIG. 9), there is a tendency towards higher levels of TNF-α, IFN-γ and IL-5, but these differences were also not significant. The levels of IL-4 and IL-2 did not differ in SLIT treated mice compared to receiving buffer. In FIGS. 8 and 9, each column represents the mean of 8 individual mice. Error bars indicate standard error of mean.

Discussion and Conclusion

Sublingual immunotherapy (SLIT) for the treatment of perennial rhinitis is safe and shows amelioration of clinical symptoms in numerous trials (5). However, relatively few studies have been performed to describe the immunological response following SLIT. The immunoregulatory mechanisms induced by SLIT administration in naive mice remains unclear, but are most likely related to those underlying the process of oral tolerance to fed antigens (18). In primed mice, SLIT administration does not appear to induce active tolerance. Previous studies have shown that once the immune system has established memory to an antigen, tolerance cannot be induced by oral administration of the antigen.

The data presented here show that SLIT treatment after priming does not induce humoral tolerance as measured by reduction of serum levels of specific antibodies. Also, no T cell proliferative response modulation has been shown by the present experiments. However, high levels of secreted IgA in BAL and NAL in mice having received SLIT suggests a redirection of the antibody response. Thus, it appears that the present treatment protocol modulates the immune response by selectively stimulating the mucosal IgA response.

Furthermore, the present study shows that the mucosal IgA antibodies are capable of inhibiting the binding of allergen to IgE antibodies. Based on this observation, it is hypothesized that sublingual desensitization to protein allergens in sensitized hosts, at least in part, is mediated through mucosal allergen-specific IgA antibodies that are able to block the binding of IgE to Phl p. Such neutralization of allergens in the microenvironment of the mucosal surfaces might prove to play a central role in the modulation of type I allergic reactions.

### Example 2

### Two-Stage Treatment Comprising Priming by Parenteral Administration and SAV by Sublingual and Peroral Administration in Mice

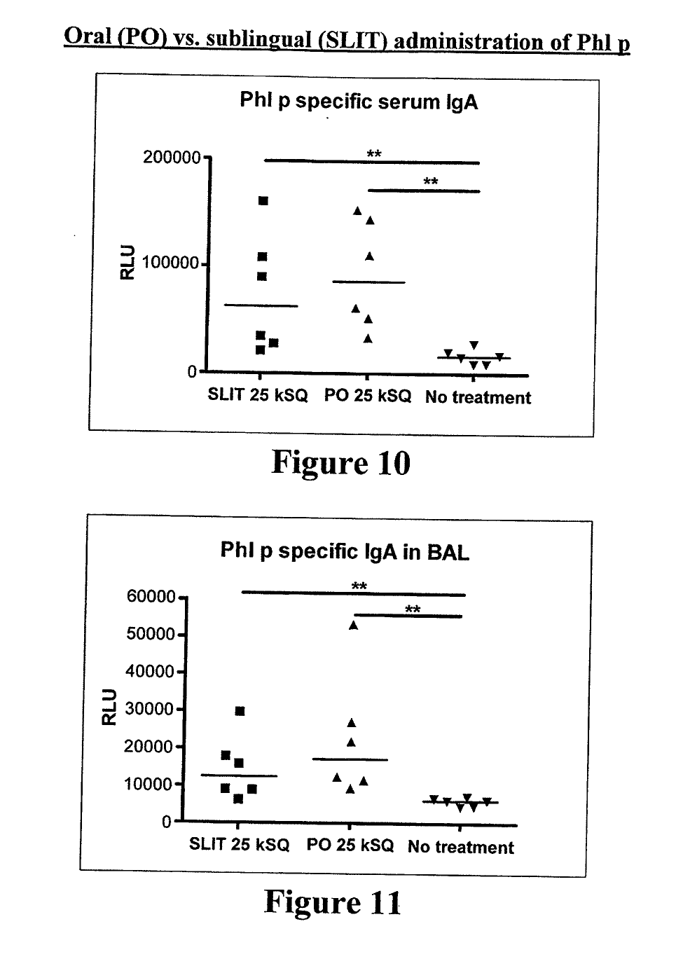

In this example the same methods and assays used in Example 1 were used. One group of mice was subjected to a treatment of a) i.p. injections (5 kSQ/alum) at weeks 0, 2 and 4 followed by b) SLIT treatment with 25 kSQ for 6 weeks. A second group of mice was subjected to a) i.p. injections (5 kSQ/alum) at weeks 0, 2 and 4 followed by b) peroral administration by gastric intubation of 25 kSQ Phl p extract in a volume of 100 μl for 6 weeks (five times per week). Finally, a control group received no treatment. IgA in serum and BAL were measured. The results are shown in FIGS. 10 and 11.

As will appear from FIGS. 10 and 11 the two treatment methods using peroral treatment and SLIT as mucosal SAV have the same level of effectiveness.

### Example 3

### Two-Stage Treatment Comprising Priming by Sublingual Administration and SAV by Parenteral Administration in Mice

Methods

Animals

Female, 6-10 week-old BALB/c mice were bred in-house and maintained on a defined diet not containing component cross reacting with antisera to Phl p. Each experimental group consisted of 8-10 animals.

Animal Experiments

Naïve mice received sublingual immunotherapy (SLIT) by buccal administration of Phl p (5 μl) daily for two to six weeks and at three different concentrations, including a buffer control. Following SLIT treatment, the mice were either sacrificed or immunized intraperitoneally (i.p.) one, two or three times with aluminiumhydroxide-adsorbed Phl p (week 6-9) and sacrificed 10 days after the last immunization. Following sacrifice blood, bronchoalveolar fluid (BAL), nasopharyngeal fluid (NAL), spleen and cervical lymph nodes were collected for analysis. Using this protocol it is possible to see whether a SLIT treatment is able to prime the immune system so as to increase the effect of the subsequent intraperitoneal treatment.

IgA, IgE, IgG, T-cell proliferation and cytokine assays were carried out as described in Example 1

Results

Antibody Response

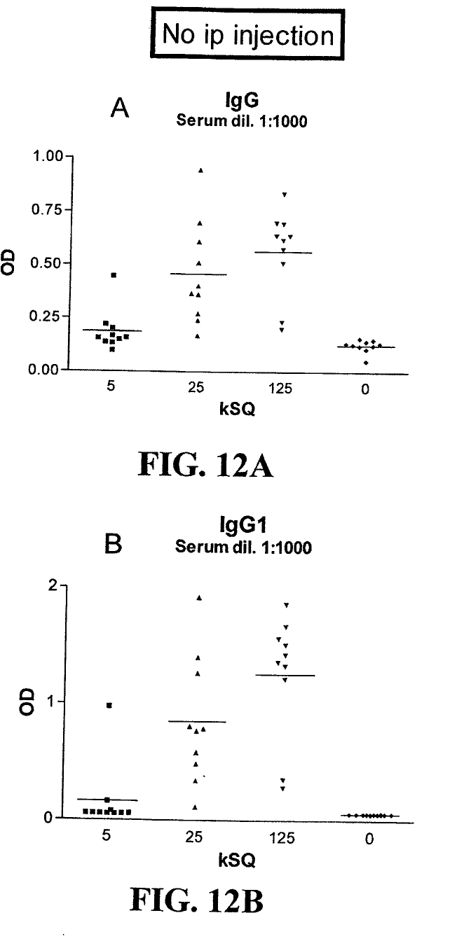

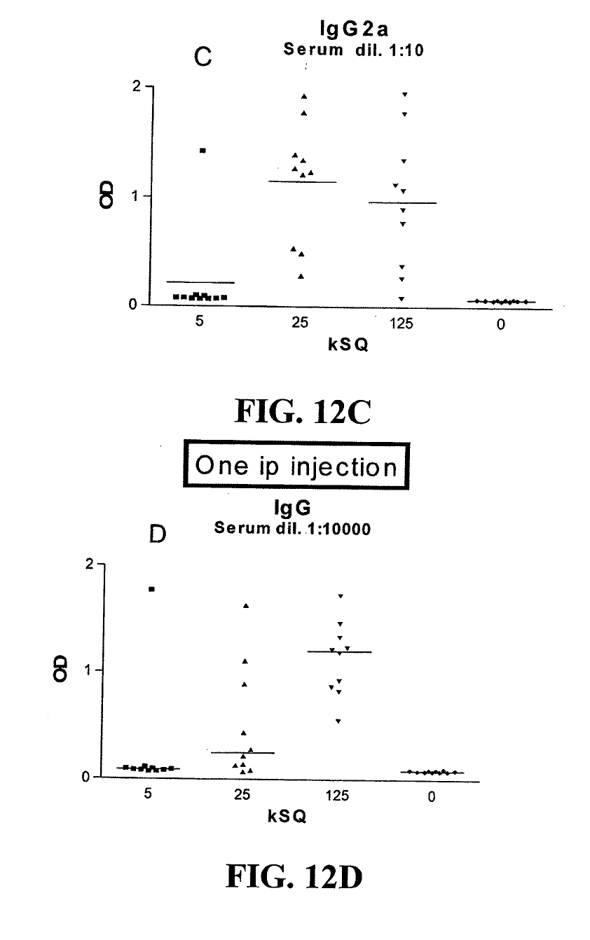

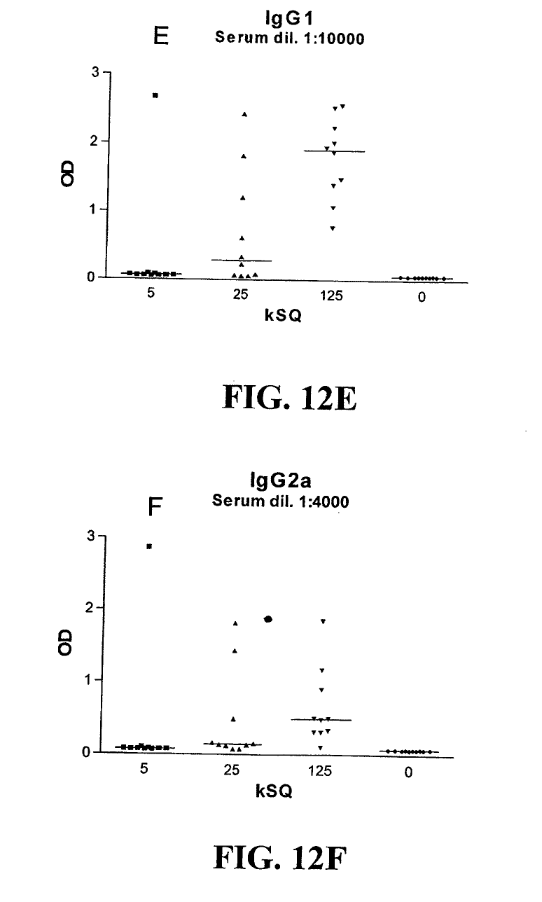

FIG. 12A-C show serum levels of Phl p specific total IgG, IgG1 and IgG2a in mice that have been treated with SLIT for six weeks. Each group of mice received daily SLIT doses of either 5, 25 or 125 kSQ, or buffer as a control. FIG. 12 D-F show serum levels of Phl p specific total IgG, IgG1 and IgG2a in mice subjected to an identical administration of SLIT followed by one i.p. immunisation with Phl p extract (5 kSQ/alum).

In the absence of i.p. injections sublingual administration of Phl p generated increasing levels of Phl p specific IgGs that were proportional to the time and dose of SLIT administration (FIG. 12A-C). Panels D-F show that SLIT followed by one i.p. injection generated IgG levels that were increased up to 40 times compared to no i.p. injection, demonstrating a priming or sensitizing effect by sublingual allergen administration. Furthermore, mice that received buffer alone as SLIT treatment does not generate significant amounts of antibodies to Phl p after one i.p. immunisation.

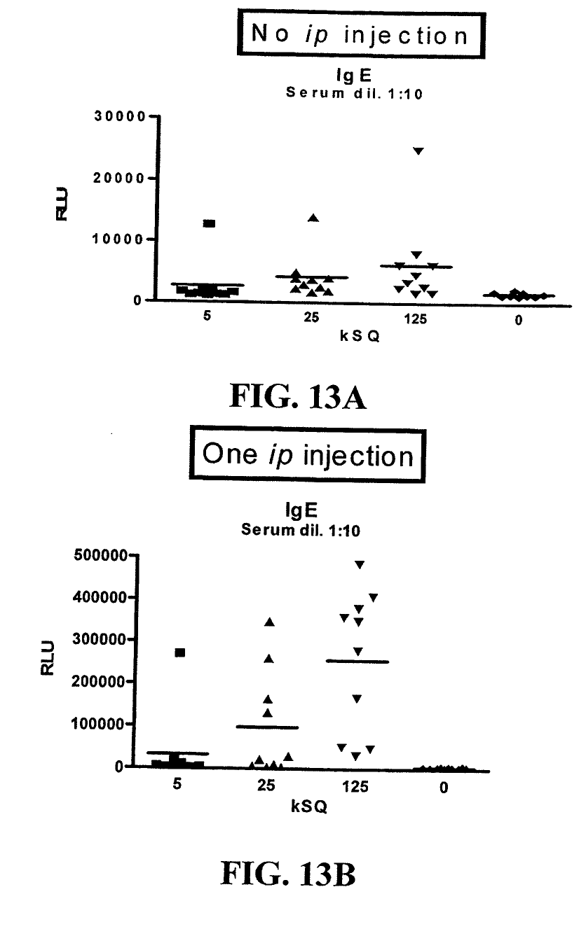

FIG. 13A shows serum levels of Phl p specific IgE in mice that have been treated with SLIT for six weeks. Each group of mice received daily SLIT doses of either 5, 25 or 125 kSQ Phl p extract, or buffer as control. FIG. 13B shows serum levels of Phl p specific IgE in mice that have been subjected to an identical administration of SLIT followed by one i.p. immunisation with Phl p extract (5 kSQ/alum). (RLU: Relative light units).

Prior to i.p. injections, low levels of specific IgE, proportional to dose and time of SLIT treatment, were observed in serum. However, as for the IgG antibodies, one i.p. injection generated IgE levels that were increased up to 60 times in mice having received Phl p-SLIT (FIG. 13). Again, a single i.p. immunisation of mice treated with buffer-SLIT does not generate significant levels of IgE.

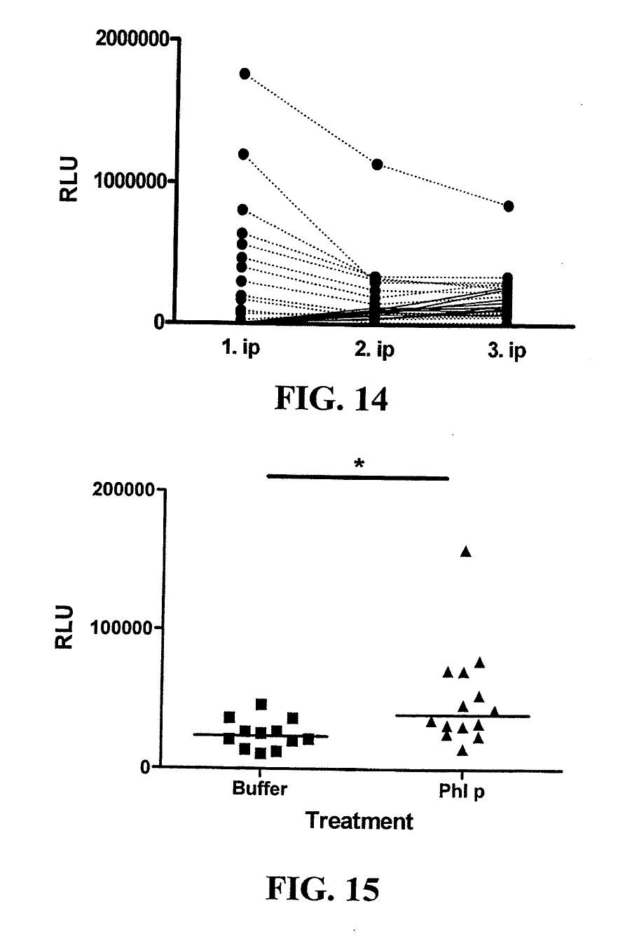

FIG. 14 shows Phl p-specific IgE levels in sera of SLIT treated (25 kSQ) (hatched lines) and buffer treated control mice (solid lines). Following SLIT treatment, the mice were immunised i.p. with Phl p extract (25 kSQ/alum) three times. One week after each immunisation the mice were bled and serum analysed for IgE levels. The first immunisation generated high IgE levels in mice having received Phl p-SLIT compared to control mice. The second and third immunisations generated increasing levels of specific IgE antibodies in the control mice whereas a strong down-regulation of the IgE-response is observed for the group of mice that received Phl p-SLIT.

The buccal administration of Phl p extract sensitises or primes the mice, since a single i.p. immunisation generates high and dose-dependent antibody levels. Although buccal administration of Phl p primes the mice as described above, repeated i.p. injections lead to a decrease in IgE levels, indicating that a specific suppression of the B cell response has been induced.

FIG. 15 shows Phl p-specific IgA levels in BAL of SLIT treated (25 kSQ) and buffer treated control mice. Following SLIT treatment, the mice were immunised i.p. with Phl p extract (25 kSQ/alum) three times. The IgA levels in BAL are significantly higher in Phl p-SLIT treated mice as compared to buffer-SLIT treated mice (P<0.05, Mann Whitney test). In contrast to the down-regulation of the IgE-response, specific IgA levels increased in BAL of mice treated with Phl p-SLIT after three i.p. immunisations.

T Cell Response

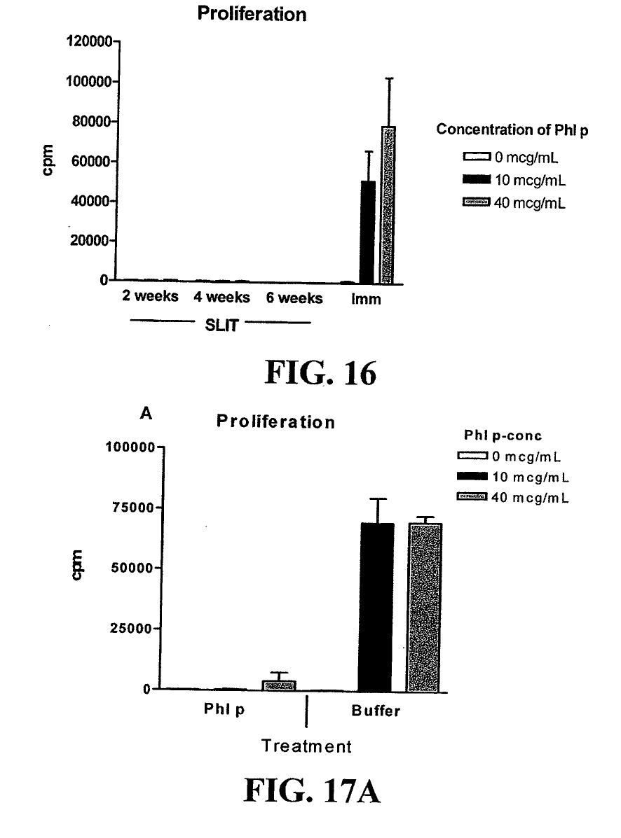

FIG. 16 shows the proliferation of spleen cells from mice treated with Phl p SLIT. Mice were given Phl p (25 kSQ) sublingually for either 2, 4 or 6 weeks. Following this, spleen cells were isolated and stimulated with Phl p in vitro at the indicated concentrations. Proliferation was measured after 6 days of incubation. As a control, spleen cells from immunised mice were included. Each column represents the mean of 6 individual mice and error bars indicate standard error of mean.

As seen in FIG. 16, SLIT given for either 2, 4 or 6 weeks did not lead to activation of spleen cells, upon allergen-specific restimulation in vitro, as the proliferation did not exceed the background values. As a positive control a strong proliferative response was seen in mice immunized with 0, 10 or 40 mcg Phl p/ml.

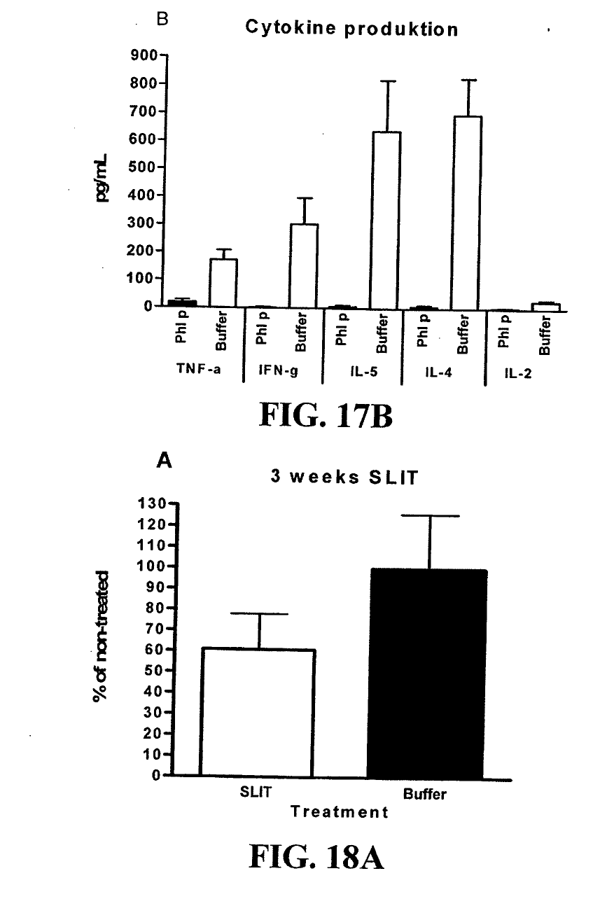

FIG. 17 shows the proliferation and cytokine production of spleen cells from mice treated with Phl p SLIT followed by one immunization. Mice were treated with either Phl p SLIT or buffer for 6 weeks, followed by one i.p. injection of alum-adsorbed Phl p. Spleen cells were isolated 8 days later and restimulated in vitro with Phl p. FIG. 17A: The proliferation measured after 6 days of incubation. FIG. 17B: Supernatants were harvested at day 5 and analyzed for TNF-α, IFN-γ, IL-4, IL-5 and IL-2. Each bar represents the mean of 8 individual mice. Error bars indicate standard error of mean.

As shown in FIG. 17, SLIT treatment with Phl p led to the induction of antigen-specific systemic tolerance, as the proliferation of spleen cells from mice that were treated with Phl p SLIT were dramatically reduced compared to mice that only received buffer. Similarly, the secretion of TNF-α, IFN-γ, IL-4 and IL-5 by the in vitro stimulated spleen cells was also reduced in mice that were treated with Phl p SLIT. IL-2 secretion was low in both SLIT and buffer treated mice.

Duration of SLIT Treatment

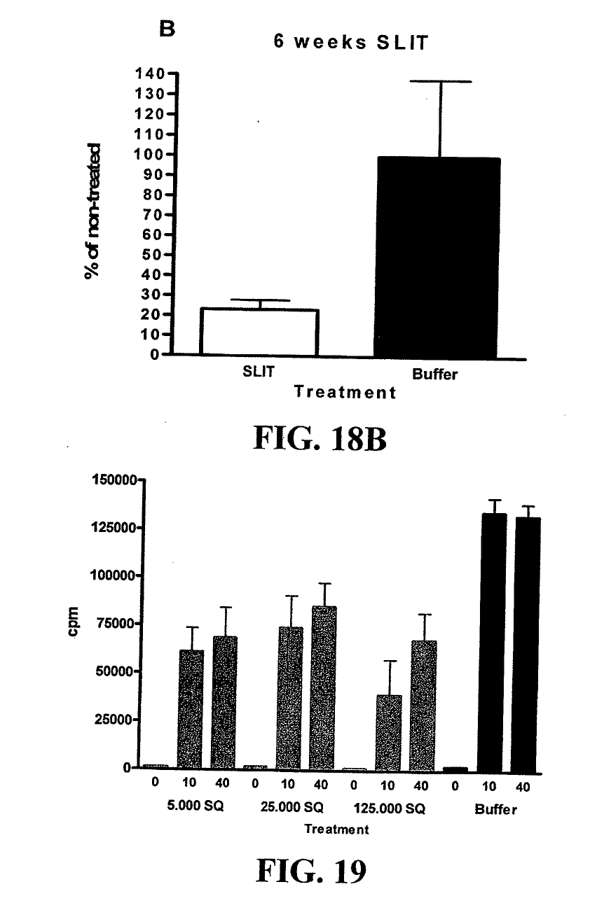

FIG. 18 shows the proliferation of spleen cells from mice treated with Phl p SLIT followed by one immunization. Mice were treated with Phl p SLIT for either 3 (FIG. 18A) or 6 (FIG. 18B) weeks followed by one i.p. injection of alum-adsorbed Phl p. Spleen cells were isolated 10 days later and restimulated in vitro with Phl p. Proliferation was measured after 6 days of incubation.

The duration of SLIT treatment seems to be important regarding the induction of T-cell tolerance. As seen in FIG. 18, SLIT-treatment for three weeks prior to immunization resulted in a less effective down-regulation of the proliferative response compared to six weeks of SLIT treatment.

Dose Response:

FIG. 19 shows the proliferation of spleen cells from mice treated with Phl p SLIT followed by one immunization. Mice were treated with either 5000 SQ, 25000 SQ or 125000 SQ for six weeks, followed by one immunization with alum-adsorbed Phl p. Spleen cells were isolated 10 days later and restimulated in vitro with Phl p. Proliferation was measured after 6 days of incubation.

Within the range of 5000-125000 SQ, the dose of Phl p used as SLIT treatment does not seem to be critical for the induction of T-cell tolerance. As seen in FIG. 19 the levels of suppression of the Phl p specific response induced by feeding 5000 SQ, 25000 SQ and 125000 SQ are similar, although there is a tendency towards a more effective suppression in mice that received 125000 SQ.

Conclusion

The results demonstrate that SLIT treatment of naïve mice primes the immune system. However, the suppression of both B and T cell responses after repeated immunizations indicate that this priming results in the induction of systemic tolerance.

1. A method of preventing or treating an allergy to or an infection by a mucosal antigenic agent in a subject comprising
a) subjecting the subject to a priming treatment by means of parenteral or adjuvant-facilitated mucosal administration of the antigenic agent to activate the systemic immune system, and
b) subjecting the subject to Specific Allergy Vaccination (SAV) by means of non-gastrointestinal mucosal administration of the antigenic agent to elicit an antigenic agent specific Ig antibody response in the mucosa of the subject.

2. A method according to claim 1, wherein the priming treatment is carried out by parenteral administration.

3. A method according to claim 1, wherein the priming treatment is carried out by adjuvant-facilitated mucosal administration.

4. A method according to claim 1 comprising the further step of performing a measurement of an activation marker to determine, whether the activation of the systemic immune system has been effected.

5. A method according to claim 4, wherein the activation marker is antigenic agent specific IgG.

6. A method according to claim 4, wherein the activation marker is antigenic agent specific IgG₄.

7. A method according to claim 1, wherein the SAV is effected by means of sublingual administration.

8. A method according to claim 7, wherein the SAV is effected using a solid dosage form.

9. A method according to claim 1 comprising the further step of performing a measurement of a mucosal marker to determine, whether an antigenic agent specific Ig antibody response in the mucosa of the subject has been elicited.

10. A method according to claim 9, wherein the Ig antibody response increases at least 5%, preferably 10%, more preferably 50%, more preferably 100% and most preferably 200% compared to the level before the priming treatment.

11. A method according to claim 9, wherein it is determined whether an antigenic agent specific Ig antibody response in the respiratory system mucosa of the subject has been elicited.

12. A method according to claim 9, wherein it is determined whether an antigenic agent specific Ig antibody response in the digestive system mucosa of the subject has been elicited.

13. A method according to claim 1, wherein the antigenic agent is a respiratory antigenic agent.

14. A method according to claim 1, wherein the antigenic agent is a digestive antigenic agent.

15. A method according to claim 1, wherein the antigenic agent is an allergen.

16. A method according to claim 15, wherein the allergen is a pollen allergen.

17. A method according to claim 1, wherein the antigenic agent is a microbial agent.

18. A method according to claim 17, wherein the microbial agent is a virus.

19. A method according to claim 17, wherein the microbial agent is a bacteria.

20. A method according to claim 1 wherein one or more additional rounds of parenteral or adjuvant-facilitated treatment is carried out subsequent to the start of the SAV by means of mucosal administration.

21. A vaccine for preventing or treating an allergy to or an infection by a mucosal antigenic agent in a subject having one or more antibodies specific for the antigenic agent by means of non-gastrointestinal mucosal administration to elicit an Ig antibody response in the mucosa of the subject.

22. Use of an allergen for preparing a vaccine for preventing or treating an allergy to or an infection by a mucosal antigenic agent in a subject having one or more antibodies specific for the antigenic agent by means of non-gastrointestinal mucosal administration to elicit an Ig antibody response in the mucosa of the subject.

23. A method of evaluating the immunological status of a subject comprising
a) measuring the level of mucosal Ig specific to an antigenic agent, and
b) using the measurement to evaluate the immunological status of the subject.

24. A method wherein the measurement of mucosal Ig is used to evaluate the effect of the method of claim 1.

25. A method wherein the level of mucosal Ig is measured two or more times during the course of the method of claim 1, and wherein the change in the measured level is used to evaluate the effect of the method.

26. A method of preventing or treating an allergy to or an infection by a mucosal antigenic agent in a subject comprising
a) subjecting the subject to a priming treatment by means of oromucosal administration of the antigenic agent to activate the mucosal immune system, and
b) subjecting the subject to Specific Allergy Vaccination (SAV) by means of parenteral or adjuvant-facilitated mucosal administration of the antigenic agent to elicit an antigenic agent specific Ig antibody response in the mucosa of the subject.

27. A method according to claim 26, wherein one or more additional rounds of treatment by mucosal administration is carried out subsequent to the start of the parenteral or adjuvant-facilitated SAV.

28. A method according to claim 26 directed to the prevention or treatment of allergy.

29. A method according to claim 28, wherein the subject does not exhibit symptoms of the allergy for which treatment is given.

30. A method according to claim 29, wherein the subject does not exhibit any symptoms of allergy.

31. A method according to 29, wherein the subject is not sensitised to the allergy for which treatment is given.

32. A method according to claim 31, wherein the subject is not sensitised to any allergy.

33. A method of screening a mutated allergen for its ability to elicit an immune response comprising
a) subjecting a subject to a priming treatment using an allergen corresponding to the wild type allergen of the mutated allergen to activate the immune system,
b) subjecting the subject to Specific Allergy Vaccination (SAV) using the mutated allergen, and
c) examining whether the mutated allergen elicits an immune response.
